(19) United States
(12) Patent Application Publication
(10) Pub. No.: US 2019/0224185 A1
(43) Pub. Date: Jul. 25, 2019

(54) PREPARATION OF (-)-COCAINE HYDROCHLORIDE

(71) Applicant: Cody Laboratories, Inc., Cody, WY (US)

(72) Inventors: Qingwei Yao, Cody, WY (US); Se-Ho Kim, Cody, WY (US)

(21) Appl. No.: 16/188,906

(22) Filed: Nov. 13, 2018

Related U.S. Application Data
(62) Division of application No. 15/981,574, filed on May 16, 2018.
(60) Provisional application No. 62/620,210, filed on Jan. 22, 2018.

(51) Int. Cl. A61K 31/46; C07D 451/02; A61P 23/02; A61K 47/12

(52) U.S. Cl. CPC A61K 31/46; C07D 451/02; A61K 47/12; A61P 23/02

(57) ABSTRACT

Efficient methods are provided for large scale production of ethyl cocaine-free cocaine hydrochloride. Compositions and methods comprising administration of cocaine hydrochloride are provided.

### CROSS-REFERENCES TO RELATED APPLICATIONS

This application is a divisional of U.S. patent application Ser. No. 15/981,574, filed May 16, 2018, which claims the benefit of U.S. Provisional Application No. 62/620,210, filed Jan. 22, 2018, the entire contents of each of which are incorporated herein by reference.

Efficient methods are provided for large scale production of ethyl cocaine-free (-)-cocaine hydrochloride.

### BACKGROUND OF THE INVENTION

Cocaine hydrochloride is an alkaloid ester used as a local anesthetic agent. Cocaine hydrochloride is used topically to produce local anesthesia of accessible mucous membranes or oral, laryngeal, and nasal cavities. It is used in both inpatient and outpatient nasal and facial surgery.

Cocaine occurs in the leaves of Erythroxylon coca and other species of Erythroxylon trees indigenous to Peru and Bolivia. The active enantiomer of cocaine is (−)-cocaine. Cocaine HCl is commercially available as colorless crystals or white, crystalline powder. The cocaine alkaloid called benzoylmethylecgonine, an ester of benzoic acid, makes up about 1.8% dry weight of Erythroxylon coca plant leaves and its related species. To obtain cocaine commercially, the coca alkaloids are hydrolyzed to form ecgonine. This is benzoylated and methylated to the base form, cocaine. Cocaine may also be produced synthetically. However, known methods for isolation or synthetic preparation of (−)-cocaine hydrochloride may suffer from low overall yield and/or undesirable impurity profiles.

2-Carbomethoxytropinone (2-CMT) has been widely utilized as a key intermediate for synthesis of cocaine and its derivatives due to its availability and functionality. For example, ecgonine methyl ester (EME), a synthetic precursor to cocaine, is directly obtained by reduction of 2-CMT with sodium-amalgam. Previous process development efforts toward synthesis of cocaine resulted in a continuous reduction of (+)-2-CMT with electrochemically generated sodium amalgam as described in U.S. Pat. No. 7,855,296, which is incorporated herein by reference in its entirety.

U.S. Pat. No. 7,855,296 discloses a method for synthesizing (+)-2-carbomethoxytropinone, or (+)-2-CMT, bitartrate which is reduced using sodium amalgam in aqueous solution with formic acid to provide a mixture of (−)-methylecgonine (EME) and pseudoecgonine methyl ester (PEM or PEME). The EME is treated with benzoyl chloride to provide (−)-cocaine as shown in FIG. 7. In the reduction step, sodium amalgam is continuously supplied from an electrolyzing unit to a reactor containing the aqueous solution of (+)-2-CMT bitartrate with addition of formic acid to maintain a pH of 5.4-5.9. Formic acid forms sodium formate—which remains soluble under aqueous reaction conditions thereby avoiding dilution of the reaction mixture. However, extended reaction times are required and the reaction is difficult to drive to completion.

Casale J. F., 1987, Forensic Sci Int 33, 275-298 discloses synthesis of cocaine enantiomers and racemic cocaine. A process is provided for batch reduction of (−)-2-CMT hydrate using 1028 g of 1.5% sodium amalgam added over 2.5 h with periodic addition of sulfuric acid to maintain pH 3-4. After stirring for another 45 min at a temperature below 5° C., and work-up, a mixture of (+)-EME and PEME was obtained. Periodic addition of water during the course of the reduction reaction was necessary to dissolve sodium sulfate salts. Following separation of mercury and workup at pH 12 with sodium hydroxide, hydrochloride salt formation and recrystallization, (+)-EME hydrochloride was obtained in a 27% yield.

Lewin et al., 1987, Journal of Heterocyclic Chemistry (1987), 24(1), 19-21 provides a practical synthesis of (+)-cocaine. Batch sodium-amalgam reduction of (−)-2-CMT was performed with periodic addition of sulfuric acid to maintain pH 3-4 at a temperature between −2 to 7° C. 1100 g of 1.5% sodium amalgam was added over a 3.5 h period and the reaction was continued for another 35 min. Water was also added during the reduction reaction to dissolve some of the salts which precipitated. After separation of the mercury, the solution was brought to pH 11 with ammonium hydroxide and extracted to provide a 2:1 mixture of (+)-EME and PEME. Hydrochloride salt formation and recrystallization afforded (+)-EME hydrochloride in a 28% yield.

Katz et al., 1992, Life Sci, 50, 1351-1361 reports comparative behavioral pharmacology and toxicology of cocaine and its ethanol-derived metabolite ethyl cocaine, also known as cocaine ethyl ester (cocaethylene). Cocaine was more potent than cocaethylene in producing increases in locomotor activity in mice; however, the two drugs were equipotent in producing convulsions, and ethyl cocaine (cocaethylene) was more potent than cocaine in producing lethality.

Casale et al., 1994, J Pharm Sci 83(8): 1186, provides analysis of pharmaceutical cocaine including ethyl cocaine (cocaethylene) and other impurities. In five commercial samples of pharmaceutical cocaine tested, ethyl cocaine (cocaethylene) was found at levels of 0.08% to 1.16% by gas chromatography-flame ionization detection after direct dissolution of the standards in ethanol-free chloroform.

Casale et al., 2008, J Forensic Sci 53(3) 661-676, disclose analysis of illicit cocaine and isolation, detection, and determination of by-products from clandestine purification of crude cocaine base with ethanol. Casale et al., 2008 reported the presence of ethyl cocaine (cocaethylene) in all exhibits that appear to have been purified.

Lange et al., 2010, European Heart J, 31(3) 271-273 investigated sudden death in cocaine abusers. The combination of cocaine and ethanol is associated with myocardial depression, decreased coronary arterial blood flow, dysrhythmias, and sudden death, all of which may be due, in part, to ethyl cocaine (cocaethylene), a pharmacologically active metabolite of cocaine that is synthesized by the liver if ethanol is present. In studies in experimental animals, Lange reported ethyl cocaine (cocaethylene) is more toxic and arrhythmogenic than either substance alone and it has a longer elimination half-life and larger volume of distribution.

An efficient, low cost, large scale method for providing (−)-cocaine hydrochloride in good yield, high enantiomeric excess, and with a minimal impurity profile is desirable. In particular, a need exists for economical and efficient methods for preparation of pharmaceutical (−)-cocaine hydrochloride with minimal toxic impurities, such as ethyl cocaine (cocaethylene).

### SUMMARY OF THE INVENTION

An efficient, low cost method for preparing (−)-cocaine hydrochloride is provided comprising reducing 2-CMT to provide EME using electrochemically generated sodium amalgam and an inorganic acid in good yield, high enantiomeric excess, and with a minimal impurity profile.

In some embodiments, a method is provided for reduction of 2-CMT to provide EME comprising exposing 2-CMT to continuously electrochemically generated sodium amalgam and sulfuric acid, wherein the method surprisingly exhibits a faster rate of reaction, and no more than 2.5% residual starting 2-CMT, as well as higher purity, and good EME/PEM ratio compared to the method of U.S. Pat. No. 7,855,296. In addition, cocaine hydrochloride prepared by the method disclosed herein comprises no more than 0.15%, 0.10%, 0.05%, 0.025%, 0.01% (100 ppm), 0.005% (50 ppm), 0.0025% (25 ppm), 0.001% (10 ppm), 0.0005% (5 ppm), or 0.0001% (1 ppm) ethyl cocaine impurity.

In some embodiments, a method of preparing (−)-cocaine or a pharmaceutically acceptable salt thereof is provided comprising exposing (+)-2-carbomethoxy-3-tropinone (2-CMT) or a salt thereof to sodium amalgam and an inorganic acid in an aqueous solution whereby at least 96%, or at least 97.5%, of the 2-CMT or salt thereof is converted to a mixture of compounds comprising (−)-ecgonine methyl ester ((−)-EME) and pseudoecgonine methyl ester (PEM); and benzoylating the (−)-EME or a pharmaceutically acceptable salt thereof to form (−)-cocaine or a pharmaceutically acceptable salt thereof. In some embodiments, at least 97.5% of the 2-CMT or salt thereof is converted to the mixture comprising (−)-EME and PEM as determined by GC area %. In some embodiments, the (+)-2-carbomethoxy-3-tropinone bitartrate is exposed to the sodium amalgam and the acid for a period of no more than 3 hours, to form the mixture of compounds comprising the (−)-EME and the PEM.

In some embodiments, a method for providing synthetic cocaine is provided comprising reducing (+)-2-CMT with sodium amalgam and an inorganic acid, comprising separating the resultant (−)-EME or pharmaceutically acceptable salt thereof from the PEM or a pharmaceutically acceptable salt thereof.

In some embodiments, a method is provided for separating (−)-EME from a crude (−)-EME and PEM compromising stirring the mixture in cyclohexane, allowing the PEM to precipitate, and filtering off the precipitated PEM.

In some embodiments, a method is provided for separating (−)-EME from PEM comprising dissolving the mixture of compounds comprising the (−)-EME and the PEM in isopropyl alcohol; adding HCl to the solution to form a mixture comprising the corresponding salts; and adding acetone to the mixture to precipitate (−) EME HCl from the mixture while leaving the PEM in the mother liquor. In some aspects, the HCl is added by addition of methanolic HCl, isopropyl alcohol HCl, HCl gas, and/or aqueous HCl in the salting step. In a particular aspect, methanolic HCl is employed. In some aspects, the salting step serves two purposes: 1) converting EME to its HCl salt; and 2) removal of any remaining PEM in the crude EME base. In some aspects, co-evaporation with isopropyl alcohol before adding acetone is performed for efficient removal of methanol.

In some embodiments, a method is provided for the removal of PEM from the EME HCl product comprising precipitating the latter from a mixture of isopropyl alcohol and acetone.

In some embodiments, a method is provided for preparing (−)-cocaine or a pharmaceutically acceptable salt thereof comprising exposing (+)-2-carbomethoxy-3-tropinone (2-CMT) bitartrate to sodium amalgam and an inorganic acid in an aqueous solution to provide (−)-EME intermediate. In some embodiments, the inorganic acid is selected from sulfuric acid, phosphoric acid, and hydrochloric acid. In a particular embodiment, the inorganic acid in the exposing step is sulfuric acid, which is employed to maintain the pH between 3.5 and 4.5. In some embodiments, the temperature of the aqueous solution during the exposing step is maintained from 5-10° C.

In some embodiments, a method is provided for providing (−)-EME, wherein the (+)-2-carbomethoxy-3-tropinone bitartrate is exposed to the sodium amalgam and aqueous sulfuric acid for a period of no more than 3 hours, to form the mixture of compounds comprising the (−)-EME and the PEM.

In some embodiments, a method for providing (−)-EME is provided wherein the (+)-2-carbomethoxy-3-tropinone bitartrate is exposed to the sodium amalgam and aqueous sulfuric acid for a period of no more than 3 hours, to form the mixture of compounds comprising the (−)-EME and the PEM, wherein the ratio of (−)-EME to PEM in the mixture is at least 1.3:1, 1.7:1, 2:1, or at least 2.4:1 or higher, by GC area %.

In some embodiments, the reduction of 2-CMT to form (−)-EME and PEM comprises continuously supplying sodium amalgam from an electrolyzing unit to the aqueous solution of (+)-2-carbomethoxytropinone or salt thereof and the inorganic acid; and continuously transferring spent amalgam from the reactor to the electrolyzing unit. In a particular embodiment, the exposing step comprises allowing an insoluble sodium salt of the inorganic acid to form during the exposing step.

In some embodiments, the exposing step comprises adding a base to the mixture of compounds comprising (−)-EME and PEM to increase the pH of the mixture to within a range from about pH 8.7 to pH 11. In some embodiments, the base in the exposing step is selected from one or more of potassium carbonate, sodium carbonate, ammonium hydroxide, magnesium hydroxide, and sodium hydroxide.

In some embodiments, isolated cocaine hydrochloride, or pharmaceutically acceptable salt thereof, is provided having not more than 0.15%, 0.10%, 0.05%, 0.01%, 0.005%, or not more than 0.001% ethyl cocaine, not more than 1.5%, 1.0%, 0.5%, 0.15%, 0.1%, 0.05% ecgonine methyl ester, or not more than 0.5%, 0.3%, 0.15%, 0.1%, 0.05% or 0.01% ecgonine, or not more than 6.5%, 5.0%, 3.0%, 1.0%, 0.5%, 0.15%, or 0.1% benzoyl ecgonine, not more than 0.2%, 0.15%, 0.1%, 0.05%, or not more than 0.01% 2′-furanoylecgonine methyl ester (FEME; 2-FEME; 2-furoyl ecgonine methyl ester), having not more than 0.5%, 0.10%, 0.05%, 0.015%, 0.01%, 0.005%, not more than 0.2%, 0.15%, 0.1%, 0.05%, or not more than 0.01% pseudococaine, not more than 0.2%, 0.15%, 0.1%, 0.05%, or not more than 0.01% dehydrococaine, not more than 0.2%, 0.15%, 0.1%, 0.05%, or not more than 0.2%, 0.1%, 0.05%, or 0.01% benzoylpseudotropine, and/or not more than 0.2%, 0.15%, 0.1%, 0.05%, or not more than 0.2%, 0.15%, 0.1%, 0.01% dehydrobenzoyltropine, by HPLC area %. In some embodiments, isolated cocaine hydrochloride, or pharmaceutically acceptable salt thereof, is provided having not more than 0.15%, 0.10%, 0.05%, 0.01%, 0.005%, or not more than 0.001% ethyl cocaine, when prepared by a method according to the present disclosure. In some aspects, isolated cocaine hydrochloride is provided devoid of detectable ethyl cocaine.

In some embodiments, a method is provided for preparing (−)-ecgonine methyl ester ((−)-EME) hydrochloride comprising exposing (+)-2-carbomethoxy-3-tropinone (2-CMT) or a salt thereof to sodium amalgam and an effective amount of an inorganic acid in an aqueous solution to maintain pH in a range from about 3 to about 4.5, wherein at least 96% of the 2-CMT or salt thereof is converted to a mixture of compounds comprising (−)-ecognine methyl ester ((−)-EME) and pseudoecgonine methyl ester (PEM) in no more than 3 hours. In some embodiments, the ratio of (−)-EME to PEM in the mixture is at least 1.3:1, 1.7:1, 2:1, 2.4:1 or higher by GC area %.

In some embodiments, the reduction of 2-CMT comprises exposing to continuously supplied sodium amalgam and an inorganic acid to form (−)-EME and PEM and an insoluble sodium salt of the inorganic acid; basification of the acidic reaction mixture to basic and extracting the crude compounds comprising the (−)-EME and the PEM with an organic solvent, preliminary removal of PEM by precipitation in cyclohexane; dissolving the crude (−)-EME still containing PEM in isopropyl alcohol and adding methanolic HCl to form a solution mixture; and adding acetone to the solution mixture to form a slurry mixture, wherein (−) EME HCl precipitates from the mixture.

In some embodiments, a pharmaceutical composition is provided comprising an effective amount of (−)-cocaine hydrochloride having not more than 0.15% ethyl cocaine, and a pharmaceutically acceptable carrier.

In some embodiments, isolated (−)-cocaine hydrochloride is provided having not more than 0.15% ethyl cocaine, prepared by a method according to the disclosure.

In some embodiments, a method for introduction of local anesthesia in a subject in need thereof is provided comprising administering a composition comprising an effective amount of (−)-cocaine hydrochloride having not more than 0.15% ethyl cocaine, and a pharmaceutically acceptable carrier.

In some embodiments, a method for introduction of local anesthesia in a subject in need thereof is provided comprising topically applying the composition comprising cocaine hydrochloride having not more than 0.15%, 0.10%, 0.05%, 0.01% (100 ppm), 0.005% (50 ppm), or 0.001% (10 ppm) ethyl cocaine to one or more mucous membranes in the subject, wherein the mucous membrane is selected from the group consisting of oral, laryngeal, and nasal mucous membranes.

In some embodiments, an aqueous topical pharmaceutical composition is provided comprising an effective amount of (−)-cocaine hydrochloride having not more than 0.15%, 0.10%, 0.05%, 0.01% (100 ppm), 0.005% (50 ppm), or 0.001% (10 ppm) ethyl cocaine, and a pharmaceutically acceptable carrier.

In some embodiments, a pharmaceutical composition is provided, comprising 2 to 20 wt/v % cocaine hydrochloride having not more than 0.15%, 0.10%, 0.05%, 0.01% (100 ppm), 0.005% (50 ppm), or 0.001% (10 ppm) ethyl cocaine; 0.05-0.2 wt/v % sodium benzoate; and 0.05-0.2 wt/v % citric acid.

In a specific embodiment, a pharmaceutical composition is provided, comprising about 4 wt/v % cocaine hydrochloride having not more than 0.15%, 0.10%, 0.05%, 0.01% (100 ppm), 0.005% (50 ppm), or 0.001% (10 ppm) ethyl cocaine; 0.85-0.15 wt/v % sodium benzoate; and 0.1-0.15 wt/v % citric acid.

In a specific embodiment, a pharmaceutical composition is provided, comprising about 10 wt/v % cocaine hydrochloride having not more than 0.15%, 0.10%, 0.05%, 0.01% (100 ppm), 0.005% (50 ppm), or 0.001% (10 ppm) ethyl cocaine; 0.85-0.15 wt/v % sodium benzoate; and 0.1-0.15 wt/v % citric acid.

In some embodiments, an aqueous topical pharmaceutical composition is provided comprising about 4% (w/v) cocaine hydrochloride that exhibits one or more of: a) estimated systemic absorption of 20 to 25% of administered dose; b) Cₘₐₓ of 130 to 150 ng/mL; c) Tₘₐₓ of 25-35 min; and/or d) apparent elimination half-life of 1-3 hours, following topical administration of about a 4 mL dose to nasal mucosa of a subject for a period of 20 minutes. In some embodiments, an aqueous topical pharmaceutical composition is provided comprising about 10% (w/v) cocaine hydrochloride and exhibits one or more of: a) estimated systemic absorption of 30 to 35% of administered dose; b) Cₘₐₓ of 420 to 450 ng/mL; c) Tₘₐₓ of 25-35 min; and/or d) apparent elimination half-life of 1-3 hours, following topical administration of about a 4 mL dose to nasal mucosa of a subject for a period of 20 minutes.

In some embodiments, isolated (−)-cocaine hydrochloride is provided for the manufacture of a medicament for introduction of local anesthesia in a human subject in need thereof, wherein the (−)-cocaine hydrochloride has not more than 0.15%, 0.10%, 0.05%, or 0.01% ethyl cocaine.

In some embodiments, a method for introduction of local anesthesia is provided comprising administering a pharmaceutical composition comprising an effective amount of (-)-cocaine hydrochloride having not more than 0.15%, 0.10%, 0.05%, or 0.01% ethyl cocaine, and a pharmaceutically acceptable carrier. In some embodiments, the pharmaceutical composition comprises 2 to 20 wt % of the (−)-cocaine hydrochloride; 0.05-0.2 wt % sodium benzoate; and 0.05-0.2 wt % citric acid. The composition may be administered prior to a surgery or a diagnostic procedure. The composition may be administered by a method comprising topically applying the composition to one or more mucous membranes in the subject, wherein the mucous membrane is selected from the group consisting of oral, laryngeal, and nasal mucous membranes. In some embodiments, the mean systemic absorption is between 20% to 35% of the total administered dose of (−)-cocaine hydrochloride.

Alternative improved methods for reduction of 2-CMT to provide EME using continuously electrochemically generated sodium amalgam were investigated. Various methods were compared to the method of U.S. Pat. No. 7,855,296, as shown in the examples.

### BRIEF DESCRIPTION OF THE DRAWINGS

FIG. 1 shows synthesis of EME HCl using electrochemically generated sodium amalgam.

FIG. 2 shows a bar graph illustrating loss of starting 2-CMT as a function of time in sodium-amalgam reduction step to form EME/PEM. Each bar represents one hour of reaction time in the various batches.

FIG. 3 shows HPLC of the purified EME HCl of Example 2 showing the EME HCl peak eluting at 9.773 min retention time at 210 nm.

FIG. 4 shows ¹H-NMR of the purified EME HCl of Example 2 formed by dissolving EME in isopropyl alcohol (IPA) and treating with methanolic HCl.

FIG. 5A shows HPLC of the purified EME HCl of Example 3 showing a single peak eluting at 9.397 min retention time at 210 nm (99.63 area %).

FIG. 5B shows GC of EME HCl prepared according to Example 3 showing single peak at essentially 100 area %

FIG. 6 shows ¹H-NMR of the purified EME HCl of Example 3 formed by dissolving EME in isopropyl alcohol (IPA) and treating with methanolic HCl.

FIG. 7 shows exemplary methods for converting (−)-EME to (−) cocaine base and subsequent hydrochloride salt formation to provide (−) cocaine hydrochloride.

FIG. 8 shows HPLC chromatogram of synthetically-derived cocaine base by HPLC method of Example 6D.

FIG. 9 shows ¹H-NMR spectrum of synthetically-derived cocaine base in CDCl₃.

FIG. 10 shows ¹³C-NMR spectrum of synthetically-derived cocaine base in CDCl₃.

FIG. 11 shows HPLC chromatogram of synthetically-derived ethyl cocaine-free cocaine hydrochloride by HPLC method of Example 6D.

FIG. 12 shows ¹H-NMR spectrum of synthetically-derived ethyl cocaine-free cocaine hydrochloride in D₂O.

FIG. 13 shows ¹³C-NMR spectrum of synthetically-derived ethyl cocaine-free cocaine hydrochloride in D₂O.

FIG. 14 shows chromatogram at 230 nm for representative resolution standard solution for related substances in naturally-derived cocaine hydrochloride HPLC method of Example 6C.

FIG. 15 shows chromatogram at 230 nm for representative cocaine hydrochloride standard solution used in naturally-derived cocaine hydrochloride HPLC method of Example 6C.

FIG. 16 shows chromatogram at 230 nm for representative sample of naturally-derived cocaine hydrochloride using HPLC method of Example 6C showing detectable ethyl cocaine impurity.

FIG. 17A shows chromatogram at 230 nm for representative resolution standard solution for related substances in synthetically-derived cocaine hydrochloride HPLC method of Example 6D.

FIG. 17B shows chromatogram at 230 nm for representative cocaine hydrochloride standard solution used in synthetically-derived cocaine hydrochloride HPLC method of Example 6D.

FIG. 17C shows chromatogram at 230 nm for representative sample of synthetically-derived cocaine hydrochloride using HPLC method of Example 6D.

FIG. 18A shows resolution chromatogram at 230 nm for representative resolution standard solution for related substances in cocaine hydrochloride HPLC method of Example 6C.

FIG. 18B shows expanded scaled chromatogram at 230 nm of representative synthetic cocaine hydrochloride lot −859, by HPLC method of Example 6C, showing absence of detectable ethyl cocaine.

FIG. 18C shows expanded scaled chromatogram at 230 nm of representative synthetic cocaine hydrochloride lot −860, by HPLC method of Example 6C, showing absence of detectable ethyl cocaine.

FIG. 18D shows expanded scaled chromatogram at 230 nm of representative synthetic cocaine hydrochloride lot −211, by HPLC method of Example 6C, showing absence of detectable ethyl cocaine.

FIG. 18E shows overlay chromatogram at 230 nm of resolution standard solution, and three representative lots of synthetic cocaine hydrochloride −859, −860 and −211, by HPLC method of Example 6C, showing absence of detectable ethyl cocaine.

FIG. 19A shows pharmacokinetic profiles: the linear mean plasma concentration-time profiles of cocaine after topical application of Cocaine Hydrochloride Topical Solution, 4% (Test-1; n=33) and 10% (Test-2; n=30), for 20 minutes by pledgets.

FIG. 19B shows pharmacokinetic profiles: the logarithmic plasma concentration profiles of cocaine after topical application of Cocaine Hydrochloride Topical Solution, 4% (Test-1; n=33) and 10% (Test-2; n=30), for 20 minutes by pledgets.

FIG. 20A shows an HPLC chromatogram of a resolution solution including benzoyl ecgonine, cocaine, ethyl cocaine, and sodium benzoate monitored at 230 nm. The HPLC method was validated to a LOD of 0.01% and a LOQ of 0.05%.

FIG. 20B shows HPLC analysis of a representative Cocaine HCl Topical Solution, 4% w/v, according to Table 11.

FIG. 20C shows HPLC analysis of a representative Cocaine HCl Topical Solution, 10% w/v, according to Table 12.

### DETAILED DESCRIPTION OF THE INVENTION

### Definitions

As used herein, the terms “administration” and “administering” refer to the act of giving a drug, or therapeutic treatment (e.g., compositions of the present application) to a subject (e.g., a subject or in vivo, in vitro, or ex vivo cells, tissues, and organs). Exemplary routes of administration to the human body can be through the mouth (oral), skin (topical or transdermal), nose (nasal or transmucosal), lungs (inhalant), oral mucosa (buccal), ear, rectal, vaginal administration. For example, methods of administration include topical administration to mucous membranes of the oral, laryngeal and nasal cavities in a subject.

The term “comprising” refers to a composition, compound, formulation, or method that is inclusive and does not exclude additional elements or method steps.

The term “consisting of” refers to a compound, composition, formulation, or method that excludes the presence of any additional component or method steps.

The term “consisting essentially of” refers to a composition, compound, formulation or method that is inclusive of additional elements or method steps that do not materially affect the characteristic(s) of the composition, compound, formulation or method.

The term “compound(s)” refers to any one or more chemical entity, pharmaceutical, drug, and the like that can be used to treat or prevent a disease, addiction, illness, sickness, or disorder of bodily function. Compounds comprise both known and potential therapeutic compounds. A compound can be determined to be therapeutic by screening using the screening methods of the present application. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of disease or condition (e.g., chronic pain).

The terms “analog” and “derivative” are interchangeable and refer to a natural or non-natural modification of at least one position of a given molecule. For example, a derivative of a given compound or molecule is modified either by addition of a functional group or atom, removal of a functional group or atom or change of a functional group or atom to a different functional group or atom (including, but not limited to, isotopes).

The term “composition(s)” refers to the combination of one or more compounds with or without another agent, such as but not limited to a carrier agent. (e.g., one or more cocaine compounds with a carrier, inert or active), making the composition especially suitable for diagnostic or therapeutic use in vitro, in vivo or ex vivo.

The term “component” refers to a constituent part of a compound, or a composition. For example, components of a composition can include a compound, a carrier, and any other agent present in the composition.

The term “effective amount” refers to the amount of a composition or compound sufficient to effect beneficial or desired results. An effective amount can be administered in one or more applications or dosages and is not intended to be limited to a particular formulation or administration route.

The term “hydrate” refers to a compound disclosed herein which is associated with water in the molecular form, i.e., in which the H—OH bond is not split, and may be represented, for example, by the formula R×H₂O, where R is a compound disclosed herein. A given compound may form more than one hydrate including, for example, hemihydrates (R×0.5H₂O), monohydrates (R×H₂O), sesquihydrates (2R×3H₂O), dihydrates (R×2H₂O), trihydrates (R×3H₂O), and the like.

The term “inhibitory” or “antagonistic” refers to the property of a compound that decreases, limits, or blocks the action or function of another compound.

The term “modulates” refers to a change in the state (e.g. activity or amount) of a compound from a known or determined state.

“Optional” or “optionally” refers to a circumstance in which the subsequently described event or circumstance may or may not occur, and that the description includes instances where said event or circumstance occurs and instances in which it does not. “Optionally” is inclusive of embodiments in which the described conditions are present and embodiments in which the described condition is not present. For example, “optionally substituted phenyl” means that the phenyl may or may not be substituted, and that the description includes both unsubstituted phenyl and phenyl wherein there is substitution. “Optionally” is inclusive of embodiments in which the described conditions are present and embodiments in which the described condition is not present.

In pharmacokinetic studies, “Cₘₐₓ” is defined as maximum observed plasma concentration that a drug achieves in a specified compartment or test area of the body after the drug has been administered and before administration of a second dose. “Tₘₐₓ” is the time of maximum observed plasma concentration; if it occurs at more than one point, Tₘₐₓ is defined as the first time point with this value. In some embodiments, mean or median Cₘₐₓ or mean or median Tₘₐₓ is determined using at least 10, at least 15, or at least 20 subjects. “T_{LQC}” is defined as time of last observed quantifiable plasma concentration. “AUC_{0-T}” is defined as cumulative area under the plasma concentration time curve calculated from 0 to T_{LQC} using the linear trapezoidal method. “AUC_{0-∞}.” is defined as area under the plasma concentration time curve extrapolated to infinity, calculated as AUC0-T+C_{LQC}/λZ, where C_{LQC} is the measured concentration at time T_{LQC}. “AUC_{0-T/∞}” is defined as relative percentage of AUC_{0-T} with respect to AUC_{0-∞}. “TLIN” is defined as time point where log-linear elimination phase begins. “λz” is defined as apparent elimination rate constant, estimated by linear regression of the terminal linear portion of the log concentration versus time curve. “Thalf” is defined as terminal elimination half-life, calculated as ln(2)/λz. “Ae” is defined as amount excreted in urine (total analyte concentration*volume of urine). “fe” is defined as fraction of dose excreted in urine (Ae/dose).

The terms “patient” or “subject” are used interchangeably and refer to any member of Kingdom Animalia. Preferably a subject is a mammal, such as a human, domesticated mammal or a livestock mammal.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ration.

The phrase “pharmaceutically-acceptable carrier” refers to a pharmaceutically-acceptable material, composition or vehicle, such as a liquid or solid filler, diluent, excipient, solvent or encapsulating material, involved in carrying or transporting the compound from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose, microcrystalline cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin (glycerol), sorbitol, mannitol and polyethylene glycol; (12) esters, such as ethyl oleate and ethyl laurate; (13) agar; (14) buffering agents, such as magnesium hydroxide and aluminum hydroxide; (15) lubricants, such as magnesium stearate, calcium stearate, zinc stearate, sorbitan monostearate, sucrose monopalmitate, glycerol dibehenate, and stearic acid; (16) alginic acid; (17) pyrogen-free sterile water; (18) isotonic saline; (19) Ringer's solution; (20) ethyl alcohol; (21) phosphate buffer solutions; (22) purified water USP; and (23) other non-toxic compatible substances employed in pharmaceutical formulations.

The term “ppm” refers to parts per million. For example, ppm may be used to refer to an amount of an impurity in an isolated compound or composition comprising a compound selected from cocaine or cocaine hydrochloride. For example, when used in reference to an impurity such as ethyl cocaine, “ppm” means parts per million of ethyl cocaine in a particular sample of an isolated compound or a composition thereof. For example, 10 ppm is equivalent to 0.001% of an impurity.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydroiodic, hydrobromic, hydrochloric, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, trifluoroacetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The term “pharmaceutically acceptable salts” includes, but is not limited to, salts well known to those skilled in the art, for example, mono-salts (e.g. alkali metal and ammonium salts) and poly salts (e.g. di- or tri-salts) of the compounds of the invention. Pharmaceutically acceptable salts of compounds of the disclosure are where, for example, an exchangeable group, such as hydrogen in —OH, —NH—, or —P(═O)(OH)—, is replaced with a pharmaceutically acceptable cation (e.g. a sodium, potassium, or ammonium ion) and can be conveniently prepared from a corresponding compound disclosed herein by, for example, reaction with a suitable base. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, hydrobromide, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The terms “treating”, “treat” or “treatment” refer to therapeutic treatment where the objective is to slow down (e.g., lessen or postpone the onset of) an undesired physiological condition, disorder or disease, or to obtain beneficial or desired results such as partial or total restoration or inhibition in decline of a parameter, value, function or result that had or would become abnormal. Beneficial or desired results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease.

The term “toxic” refers to any detrimental or harmful effects on a subject, a cell, or a tissue as compared to the same cell or tissue prior to the administration of the toxicant.

The term “purified” or “to purify” or “substantially purified” refers to the removal of inactive or inhibitory components or impurities (e.g., contaminants) from a composition to the extent that 10% or less, e.g., 10%, 9%, 8%, 7%, 6%, 5%, 4%, 3%, 2%, 1%, 0.5%, 0.15%, 0.1%, 0.05% (500 ppm), 0.025% (250 ppm), 0.01% (100 ppm), 0.005% (50 ppm), 0.0025% (25 ppm), 0.001% (10 ppm), 0.0005% (5 ppm), 0.0001% (1 ppm)_or less, of the composition is not active compounds or pharmaceutically acceptable carrier.

The term “isolated” refers to the separation of a material from at least one other material in a mixture or from materials that are naturally associated with the material. For example, a compound synthesized synthetically is separated from a starting material or an intermediate.

The term “alkyl” refers to a branched or unbranched saturated hydrocarbon group of 1 to 24 carbon atoms. Preferred “alkyl” groups herein contain 1 to 16 carbon atoms; i.e. C₁₋₁₆ alkyl. Examples of an alkyl group include, but are not limited to, methyl, ethyl, propyl, iso-propyl, butyl, iso-butyl, secondary-butyl, tertiary-butyl, pentyl, iso-pentyl, neo-pentyl, hexyl, iso-hexyl, 3-methylpentyl, 2,3-dimethylbutyl, neo-hexyl, heptyl, octyl, nonyl, decyl, undecyl, dodecyl, tridecyl, tetradecyl, pentadecyl, and hexadecyl. Most preferred are “lower alkyl” which refer to an alkyl group of one to six, more preferably one to four, carbon atoms. The alkyl group may be optionally substituted with an acyl, amino, amido, azido, carboxyl, alkyl, aryl, halo, guanidinyl, oxo, sulfanyl, sulfenyl, sulfonyl, heterocyclyl, heteroaryl, or hydroxyl group.

The term “alkali metal salt” or “alkali metal hydroxide” refers to metallic salts, such as halide salts, or hydroxides, respectively, that include, but are not limited to, appropriate alkali metal (group 1) salts, e.g., lithium, sodium, potassium, rubidium, cesium, and francium salts or hydroxides.

The term “alkaline earth metal” (group 2) salts, hydroxides or oxides refers to salts, such as halide salts, oxides or hydroxides of, e.g., beryllium, magnesium, calcium, strontium, barium, and radium. Salts of other physiologically acceptable metals may be employed.

The term “alcohol” refers to “hydroxy” or “hydroxyl” and refers to the substituent —OH.

The term “amino alcohol” refers to a functional group containing both an alcohol and an amine group. As used herein, “amino alcohols” also refers to amino acids as defined above having a carbon bound to an alcohol in place of the carboxylic acid group. In exemplary embodiments, the term “amino alcohol” refers to an amino alcohol as defined above wherein the amine is bound to the carbon adjacent to the alcohol-bearing carbon. In exemplary embodiments, “amino alcohol” refers to an amine and alcohol-containing moiety as described above containing 1, 2, 3, 4, 5, 6, 7, 8, 9, 10, 11 or 12 carbon atoms (i.e., C₁₋₁₂ amino alcohol). Examples of amino alcohols include, but are not limited to, ethanolamine, heptaminol, isoetarine, norepinephrine, propanolamine, sphingosine, methanolamine, 2-amino-4-mercaptobutan-1-ol, 2-amino-4-(methylthio)butan-1-ol, cysteinol, phenylglycinol, prolinol, 2-amino-3-phenyl-1-propanol, 2-amino-1-propanol, cyclohexylglycinol, 4-hydroxy-prolinol, leucinol, tert-leucinol, phenylalaninol, α-phenylglycinol, 2-pyrrolidinemethanol, tyrosinol, valinol, serinol, 2-dimethylaminoethanol, histidinol, isoleucinol, leucinol, methioninol, l-methyl-2-pyrrolidinemethanol, threoninol, tryptophanol, alaninol, argininol, glycinol, glutaminol, 4-amino-5-hydroxypentanamide, 4-amino-5-hydroxypentanoic acid, 3-amino-4-hydroxybutanoic acid, lysinol, 3-amino-4-hydroxybutanamide, and 4-hydroxy-prolinol.

The term “amino acid” refers to a group containing a carboxylic acid and an amine bound to the carbon atom immediately adjacent to the carboxylate group, and includes both natural and synthetic amino acids. Examples of amino acids include, but are not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. The carboxyl is substituted with H, a salt, ester, alkyl, or aralkyl. The amino group is substituted with H, acyl, alkyl, alkenyl, alkynyl, carboxyl, cycloalkyl, aralkyl, or heterocyclyl.

The term “ether” refers to the group —R′—O—R″ wherein R′ and R″ as used in this definition are independently hydrogen, alkyl, alkenyl, alkynyl, carbocyclic, heterocylic, aryl, or aralkyl, and R′ can additionally be a covalent bond attached to a carbon.

The term “halogen” refers to a fluorine, chlorine, bromine or iodine atom.

The term “halide” or “halo” refers to a functional group containing an atom bond to a fluorine, chlorine, bromine or iodine atom. Exemplary embodiments disclosed herein may include “alkyl halide,” “alkenyl halide,” “alkynyl halide,” “cycloalkyl halide,” “heterocyclyl halide,” or “heteroaryl halide” groups. In exemplary embodiments, “alkyl halide” refers to a moiety containing a carbon-halogen bond containing 1, 2, 3, 4, 5, 6, 7, 8, 9 or 10 carbon atoms (i.e., C₁₋₁₀ alkyl halide). Examples of an alkyl halide group include, but are not limited to, fluoromethyl, fluoroethyl, chloromethyl, chloroethyl, bromomethyl, bromoethyl, iodomethyl and iodoethyl groups. Unless otherwise indicated, any carbon-containing group referred to herein can contain one or more carbon-halogen bonds. By way of non-limiting example, a Ci-alkyl group can be, but is not limited to, methyl, fluoromethyl, difluoromethyl, trifluoromethyl, chloromethyl, dichloromethyl, trichloromethyl, bromomethyl, dibromomethyl, tribromomethyl, iodomethyl, diiodomethyl, triiodomethyl, chlorofluoromethyl, dichlorofluoromethyl, and difluorochloromethyl.

Regioisomers or regio-isomers are structural isomers that are positional isomers consisting of different compounds with the same molecular formula comprising one or more functional group(s) or other substituent(s) that change(s) position on a parent structure.

Enantiomers are defined as one of a pair of molecular entities which are mirror images of each other and non-superimposable.

Diastereomers or diastereoisomers are defined as stereoisomers other than enantiomers. Diastereomers or diastereoisomers are stereoisomers not related as mirror images. Diastereoisomers are characterized by differences in physical and chemical properties.

Organic acid refers to an acid comprising at least one carbon atom in its chemical structure. Non-limiting examples of organic acids include formic acid, trifluoroacetic acid, oxalic acid, succinic acid, citric acid, acetic acid, ethanesulfonic acid, toluenesulfonic acid, and tartaric acid.

Inorganic acid refers to an acid that does not contain at least one carbon atom in its chemical structure. Non-limiting examples of inorganic acids include sulfuric acid, phosphoric acid, hydrochloric acid, hydrobromic acid, nitric acid, tetrafluoroboric acid, and hexafluorophosphoric acid.

Unless otherwise specified, when a compound having “not more than x %” or “not more than y ppm” of an impurity is disclosed, the x % or y ppm refers to the area of the principle peak in a chromatogram obtained with the reference compound. Unless otherwise specified, the chromatogram is an HPLC chromatogram.

The term “cocaine” refers to (L)-cocaine, (−)-cocaine, also known as methyl (1R,2R,3S,5 S)-3-(benzoyloxy)-8-methyl-8-azabicyclo[3.2.1]octane-2-carboxylate, synonyms include (1R,2R,3S,5S)-2-methoxycarbonyltropan-3-yl benzoate, and 3beta-hydroxy-1 alphaH, 5alphaH-tropane-2beta-carboxylic acid methyl ester benzoate.

The term “ethyl cocaine” or “ethylcocaine” or “cocaethylene” or “cocaine ethyl ester” or “ethylbenzoylecgonine” may be used interchangeably and refer to ethyl (1R,2R,3S,5S)-3-(benzoyloxy)-8-methyl-8-azabicyclo[3.2.1]octane-2-carboxylate. Ethyl cocaine is the ethyl ester of benzoylecgonine and is structurally similar to cocaine which is the methyl ester of benzoylecgonine.

The term “cocaine hydrochloride” refers to (−)-cocaine HCl, (−)-cocaine hydrochloride, (L)-cocaine HCl, or (L)-cocaine hydrochloride, also known as methyl (1R,2R,3S,5S)-3-(benzoyloxy)-8-methyl-8-azabicyclo[3.2.1]octane-2-carboxylate hydrochloride; or (1R,2R,3 S,5 S)-methyl 3-(benzoyloxy)-8-methyl-8-azabicyclo[3.2.1]octane-2-carboxylate hydrochloride. Cocaine hydrochloride is a synthetic tropane alkaloid ester, local anesthetic, which occurs as colorless to white crystals or white crystalline powder. The structural formula for cocaine hydrochloride is as follows.

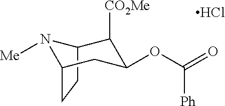 [SMILES: COC(=O)C1C(OC(=O)c2ccccc2)CC2CCC1N2C ; Cl]

The term “2-CMT” refers to 2-carbomethoxy-3-tropinone, also known as 2-carbomethoxytropinone, also known as methyl (1S,5R)-8-methyl-3-oxo-8-azabicyclo[3.2.1]octane-4-carboxylate. 2-CMT may occur as a racemic mixture of (+)-2-CMT and (−)-2-CMT, or as a particular enantiomer. Unless otherwise specified, 2-CMT refers to (+)-2-CMT. (+)-2-CMT or a salt thereof may be obtained commercially, or by any method known in the art. For example, Kuznetsov U.S. Pat. No. 7,855,296 resolves racemic (+)-2-CMT with (+)-tartaric acid to obtain (+)-2-CMT bitartrate.

The term “EME” refers to ecgonine methyl ester, also known as methylecgonine, or methyl (1R,2R,3 S,5S)-3-hydroxy-8-methyl-8-azabicyclo[3.2.1]octane-2-carboxylate. Unless otherwise specified “EME” refers to (−)-EME.

The terms “PEM” or “PEME” refers to pseudoecgonine methyl ester, or pseudo-methylecgonine, or methyl (1R,2S,3 S,5 S)-3-hydroxy-8-methyl-8-azabicyclo[3.2.1]octane-2-carboxylate.

The term “ethyl cocaine-free cocaine hydrochloride” refers to isolated cocaine hydrochloride wherein the ethyl cocaine impurity is not detected in an HPLC method having a limit of detection (LOD) of 100 ppm ethyl cocaine or lower. In some embodiments, the ethyl cocaine-free cocaine hydrochloride has no more than 0.15%, 0.10%, 0.05%, 0.01%, 0.005%, or 0.001% (10 ppm) ethyl cocaine by HPLC. In some aspects, ethyl cocaine-free cocaine hydrochloride includes no more than 100 ppm, 50 ppm, 25 ppm, 10 ppm, 0.0005% (5 ppm), 0.0002% (2 ppm), or no more than 0.0001% (1 ppm) ethyl cocaine, or is preferably devoid of detectable ethyl cocaine.

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” refers to and encompasses any and all possible combinations of one or more of the associated listed items.

The term “about,” as used herein when referring to a measurable value such as an amount of a compound, dose, time, temperature, and the like, is meant to encompass variations of 10%, 5%, 1%, 0.5%, or even 0.1% of the specified amount.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms, including technical and scientific terms used in the description, have the same meaning as commonly understood by one of ordinary skill in the art to which this disclosure belongs. In the event of conflicting terminology, the present specification is controlling.

An efficient, low cost method is provided herein for preparing isolated (−)-cocaine hydrochloride on a large scale comprising reducing 2-CMT to provide EME and PEM, producing EME HCl, benzoylation of the EME to form cocaine base, hydrochloride salt formation to provide (−)-cocaine hydrochloride, and isolating the (−)-cocaine hydrochloride.

The disclosure provides an improved method for making a key intermediate in the synthesis of isolated cocaine hydrochloride. EME is produced by reducing (+)-2-CMT with sodium amalgam and sulfuric acid, without adding water to solubilize sodium sulfate by-product during the reaction. Use of sulfuric acid offers advantages as an acid being used for pH control leading to the reaction rate enhancement and high EME/PEM ratios wherein the reducing step is performed in no more than 3 hours. These factors contributed to producing the final EME HCl with high yield and purity (28-31% yield and 98.0-99.7% purity).

When using formic acid in the reduction reaction, slow conversion of 2-CMT to EME/PEM was observed in the mid-late stage of reaction. The high-water solubility of sodium formate formed during the reduction process could contribute to an increase in solution viscosity that tends to slow down the rate of conversion of residual 2-CMT, especially at a late stage of the reaction. Due to the formation of a formic acid buffer in the reduction reaction containing formic acid and sodium formate, a large amount of sodium carbonate was required to raise the pH of the mixture to 9-10 in the basification step and troublesome gas bubbles were also formed.

In some embodiments, a method is provided for providing key intermediate (−)-EME HCl in good yield, high enantiomeric excess, and with a minimal impurity profile, comprising exposing (+)-2-CMT to electrochemically generated sodium amalgam and an inorganic acid.

Prior art batch syntheses of (+)-EME using sodium amalgam and sulfuric acid were performed by others including Lewin 1987 and Casale et al. 1987; however, significant amounts of water were required to be added during the reduction reaction in order to solubilize the relatively insoluble sodium sulfate by-product. This process was believed to be unwieldy, particularly in a large scale format, at least due to the need to remove mercury impurities prior to work-up.

Previous process development efforts toward large scale synthesis of cocaine resulted in a process comprising continuous reduction of 2-CMT to form a 3:2 mixture of EME and PEM with electrochemically generated sodium amalgam and formic acid as disclosed in Kuznetsov U.S. Pat. No. 7,855,296, which is incorporated herein by reference in its entirety. However, the Kuznetsov process was found to be somewhat difficult to drive to completion, and required at least 4 to 6 hours or more to arrive at 90 to 95% consumption of the 2-CMT starting material.

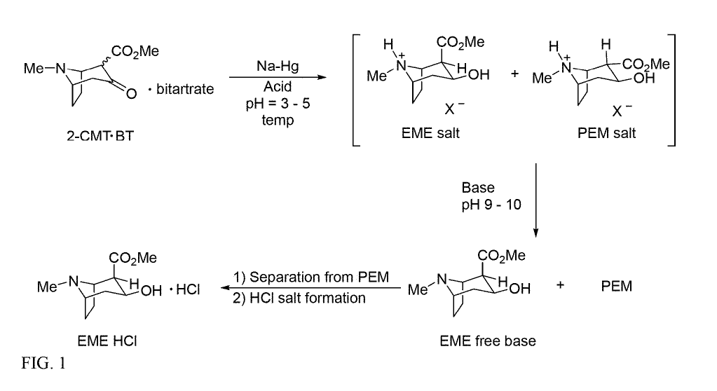

In some embodiments, a method is provided for reducing (+)-2-carbomethoxytropinone using continuously supplied sodium amalgam and an inorganic acid to form a mixture of compounds comprising (−)-methylecgonine (EME) and pseudo-methylecgonine (PEM) in a ratio of at least 1.3:1, 1.5:1, 1.7:1, 2:1, or at least 2.4:1. The method is performed as outlined in the first step of FIG. 1. FIG. 1 shows synthesis of key intermediate EME HCl by reduction of 2-CMT using electrochemically generated sodium amalgam.

Starting Material (+)-2-Carbomethoxy-3-Tropinone

In some embodiments, the starting material 2-carbomethoxy-3-tropinone, or (+)-2-CMT, may be produced by any method known in the art, or may be purchased commercially. For example, (+)-2-CMT may be produced by a method similar to that of Casale 1987, Carroll 1982, or Kuznetsov U.S. Pat. No. 7,855,296, each of which are incorporated herein by reference. For example, Casale 1987, Forensic Sci Int, 33, 275-298, prepares (−)-2-CMT by first converting acetonedicarboxylic acid into its anhydride and then preparing the methyl ester from the anhydride. The monomethyl ester of acetonedicarboxylic acid is reacted with methylamine and succindialdehyde via the Mannich condensation to yield (−)-2-CMT. Carroll 1982, J Org Chem, 47, 13-19, prepares 2-CMT by addition of 3-tropinone (Hooker) in dry cyclohexane to a mixture of anhydrous cyclohexane, NaH and dimethyl carbonate under nitrogen. After 1.75 h under reflux, the reaction mixture was cooled and water was added and the cyclohexane layer was extracted with additional water. It is preferable that the 2-CMT starting material is prepared by a method that does not employ ethanol. The combined aqueous extract was extracted with CHCl₃ and combined CHCl₃ extract was washed with saturated aqueous NaCl and dried over Na₂SO₄ overnight. The solvent was evaporated after removal of the drying agent, leaving a yellowish oil as (+/−)-2-CMT. The 2-CMT enantiomers may be resolved by any method known in the art, for example by formation and selective crystallization of tartaric acid salts.

Kuznetsov U.S. Pat. No. 7,855,296 discloses a method for preparing (+)-2-carbomethoxytropinone (2-CMT) bitartrate. 2,5-Dimethoxytetrahydrofurane is added to 0.2 N sulfuric acid and stirred at ambient temperature for 2.5 h to give a solution of succindialdehyde. Acetonedicarboxylic acid anhydride is added to methanol and stirred to form acetone dicarboxylic acid monomethyl ester. The succindialdehyde solution is combined with aqueous citric acid and the acetonedicarboxylic acid monomethyl ester in methanol. Methylamine hydrochloride was added and stirred at ambient temperature for 16 hours. Then the mixture was treated with aqueous NaOH and worked up to obtain racemic 2-CMT. Kuznetsov resolves racemic-2-carbomethoxytropinone in a first organic solvent not miscible with water to a solution of (+)-tartaric acid in water to create an aqueous phase having diastereomeric salts of 2-carbomethoxytropinone with (+)-tartaric acid; adding a second organic solvent miscible with water to the aqueous phase to obtain crystalline (+)-2-carbomethoxytropinone bitartrate.

Sodium Amalgam Reduction Step

Methods are provided for reducing the starting material 2-CMT with sodium-amalgam to form (−)-EME, a synthetic precursor to cocaine, as outlined in FIG. 1.

In one example, during the electrolysis operation, sodium amalgam (Na—Hg; Na-amalgam) is constantly made by electrolysis and pumped to the reactor where it reacts with the (+)-2-CMT. Spent amalgam depleted of sodium flows back to the electrolyzing unit where it is replenished with sodium. The process continues until substantially all, or at least 96%, of the (+)-2-CMT is converted. Thus, two separate steps: preparation of sodium amalgam and reduction of 2-carbomethoxytropinone are combined into a single uninterrupted process. In some embodiments, the reducing step comprises exposing the (+)-2-CMT to an aqueous solution comprising sodium amalgam and an inorganic acid, wherein the sodium amalgam is produced continuously over at least a portion of, a substantial portion of, or over the full time course of the reaction. In some embodiments, the reducing step comprises using electrochemically generated amalgam and an acid.

Since the Na-amalgam reduction is strongly affected by the pH of the reaction, an acid should be used to maintain the desired pH (3-5) of the reaction as shown in FIG. 1. Several organic acids (e.g., formic acid, trifluoroacetic acid) and inorganic acids (e.g., phosphoric acid, sulfuric acid) as well as acid resin can be used for this purpose. In some embodiments, the acid may be an organic acid, or an inorganic acid. In some embodiments, the inorganic acid is selected from sulfuric acid, phosphoric acid, hydrochloric acid, hydrobromic acid, nitric acid, tetrafluoroboric acid, and hexafluorophosphoric acid. In a specific embodiment, the inorganic acid is sulfuric acid. In some embodiments, the organic acid is selected from formic acid, acetic acid, propionic acid, trifluoroacetic acid, chloroacetic acid, oxalic acid, succinic acid, citric acid, ethanesulfonic acid, toluenesulfonic acid, and tartaric acid

In the method, the sodium amalgam is continuously supplied from an electrolyzing unit to a reactor containing the aqueous solution of (+)-2-carbomethoxytropinone bitartrate and an acid. The spent amalgam may further be continuously removed from the reactor and transferred to the electrolyzing unit for regeneration. For example, the preparation of (−)-methylecgonine may utilize a reactor connected via the bottom drain to an electrolyzing unit. In an embodiment, the reactor is a fiberglass reactor equipped with a cooling coil and an efficient mechanical stirrer. In addition, a mechanism is provided that transfers amalgam generated in the electrolyzing unit to the reactor. Such a transfer mechanism may be automated to continuously transfer the amalgam to the reactor.

In an embodiment, the process is continued until the conversion of 2-carbomethoxytropinone into a mixture of compounds comprising methylecgonine (EME) and pseudo-methylecgonine (PEM) exceeds 96% (for example, as determined by gas chromatography). The time required to achieve this conversion will vary depending on the exact equipment used as well as such variables as the current supplied in the electrolysis unit, the amount of mercury used, and the pH. Alternatively, the electrolysis could be performed for a predetermined period of time or until some predetermined conversion threshold is reached.

In some embodiments, the reducing step is performed over a period of no more than 4 hours, or no more than 3 hours to provide over 96%, over 97%, over 97.5%, or over 98% conversion of (+)-2-CMT to a mixture of compounds comprising (−)-EME and PEM.

In some embodiments, the disclosure provides a method comprising reduction of 2-CMT to provide EME and PEM with continuously generated sodium amalgam carried out at a temperature of from 5 to 15° C., or 5 to 10° C.

In some embodiments, the disclosure provides a method comprising reduction of 2-CMT to provide EME and PEM with continuously generated sodium amalgam carried out without addition of water to dissolve sodium sulfate by-product.

In some embodiments, the disclosure provides a method for reduction of 2-CMT to provide EME and PEM comprising exposing the 2-CMT to continuously generated sodium amalgam at a pH of from 3.5 to 4.5.

In some embodiments, the disclosure provides a method comprising reduction of 2-CMT to provide EME and PEM over a period of from 2 to 18 hours, 2.5 to 5 hours, or no more than 3 hours, to provide a ratio of EME to PEM of greater than 1.3:1, 1.7:1, or 2.4:1, or from 1.3:1 to 3.2:1, or from 2.4:1 to 3.2:1.

Improved methods are provided for producing key intermediate (−)-EME HCl from 2-CMT. Three groups of reaction conditions were compared as shown in Table 1. As shown in Example 1, the first group (Experiment A) used sulfuric acid in a first test procedure, the second group (Experiment B) used formic acid in a second test procedure, but otherwise employed the same conditions as Experiment A, and the third group (Experiment C) of experiments were based on comparative process of Kuznetsov U.S. Pat. No. 7,855,296, in which formic acid was found to be a suitable choice of acid because of the high water solubility of the corresponding conjugate base (sodium formate).

The three groups of experiments include a two-step process involving reduction of 2-CMT followed by HCl salt formation as shown in FIG. 1. Key parameters most considered were pH, temperature, acid, reaction rate, EME/PEM ratios, extraction efficacy, yield and purity. During the study, the efficiency of three group experiments (A, B, and C) was systematically evaluated with respect to these parameters and we sought to understand the differential effect of sulfuric acid and formic acid on the outcome of the reaction. The resulting data are summarized in Tables 2-3 and all aspects of experiments are subsequently discussed in detail.

The experiments were performed in a 500 mL-jacketed reactor which is connected to an electrolysis cell being set up with power supply. The electrolysis cell is designed to contain approximately 4.3 kg mercury and 600 mL of 50 wt % NaOH solution. Each group of the experiments was carried out in triplicate. Experimental design is shown in Table 1.

TABLE 1
Experimental design and some key reaction parameters

| | Experimental group | | |
|---|---|---|---|
| | A | B | C |
| Method | Test | Test | Comparative |
| | Process 1 | Process 2 | Process |
| | | | U.S. Pat. No. |
| | | | 7,855,296 |
| Number of | 3 (A1-A3) | 3 (B1-B3) | 3 (C1-C3) |
| batches^{a} | | | |
| Acid being used | Sulfuric acid | Formic acid | Formic acid |
| for pH control | | | |
| pH of the reaction | 3.5-4.5 | 3.5-4.5 | 4.5-5.5 |
| Reaction | 5-10° C. | 5-10° C. | 0-5° C. |
| temperature | | | |
| Basification | Na₂CO₃ | Na₂CO₃ | NH₄OH |
| (pH 9-10) | | | |
| Extraction | CH₂Cl₂ | CH₂Cl₂ | CHCl₃ |
| solvent (volume) | (230 mL) | (230 mL) | (536 mL) |
| HCl salt | c-HCl | c-HCl | HCl |
| formation | (12 M in water) | (12 M in water) | (2 M in ether) |

^{a}Reaction scale: 2-CMT bitartrate (30.56 g, 87.99 mmol)

Detailed experimental protocols for representative A, B, and C batches are shown in Example 1.

Comparative reaction times, GC profiles after sodium-amalgam reduction and EME/PEM ratios are shown in Table 2.

TABLE 2
Reaction time, GC profiles after sodium-amalgam reduction and
EME/PEM ratios.

| | | Batch | | | | | | | | |
|---|---|---|---|---|---|---|---|---|---|---|
| | | A1 | A2 | A3 | B1 | B2 | B3 | C1 | C2 | C3 |
| Reaction time | | 3 h | 3 h | 3 h | 4 h | 6 h | 5 h | 6 h | 6 h | 6 h |
| GC^{a} | 2-CMT | 2.3 | 2.3 | 1.0 | 4.2 | 6.3 | 5.0 | 9.7 | 5.5 | 7.0 |
| (% | EME | 71.9 | 69.9 | 74.7 | 55.7 | 57.8 | 68.4 | 57.7 | 61.5 | 60.3 |
| area) | PEM | 25.8 | 28.8 | 24.3 | 35.2 | 32.1 | 26.1 | 32.3 | 33.3 | 32.7 |
| | Impurity 1 | — | — | — | 4.9 | 3.5 | — | — | — | — |
| | Impurity 2 | — | 0.4 | — | — | 0.4 | 0.2 | — | — | — |

| | | | | | | | | | |
|---|---|---|---|---|---|---|---|---|---|
| Ratio | 2.9/1 | 2.4/1 | 3.1/1 | 1.6/1 | 1.8/1 | 2.6/1 | 1.8/1 | 1.9/1 | 1.8/1 |
| (EME/PEM) | | | | | | | | | |

^{a}Analyzed after completion of Na-amalgam reduction

The GC peak areas for batches A1-A3, B1-B3 and C1-C3, shown in Table 2, were compared after completion of Na-amalgam reduction. As can be seen in Table 2, use of sulfuric acid and without adding water during the reduction reaction to dissolve sodium sulfate by-product, resulted in less than 2.5% residual 2-CMT starting material after 3 h reaction time as revealed by GC analysis. This is in contrast to the comparative patented process which resulted in greater than 5.5% residual CMT after 6 h reaction time.

Yield and purity of each batch of EME, and EME HCl are shown in Table 3.

TABLE 3
Yield and purity of each batch

| | | test | | | test | | | comparative | | |
|---|---|---|---|---|---|---|---|---|---|---|
| | | Batch | | | | | | | | |
| | | A1 | A2 | A3 | B1 | B2 | B3 | C1 | C2 | C3 |
| Na—Hg | Amt | 9.08 g | 10.23 g | 9.26 g | 9.18 g | 9.99 g | 8.80 g | 13.95 g | 12.73 g | 11.65 g |
| reduction | (crude) | | | | | | | | | |
| | Y_{crude}¹ | 52% | 58% | 53% | 52% | 57% | 50% | 80% | 73% | 66% |
| | Y_{EME}² | 37% | 41% | 39% | 29% | 33% | 34% | 46% | 45% | 40% |
| Salting | Amt | 6.04 g | 6.33 g | 5.74 g | 5.2 g | 4.88 g | 4.47 g | 5.97 g | 6.16 g | 5.77 g |
| step | (EME | | | | | | | | | |
| | HCl) | | | | | | | | | |
| | Y_{salting}² | 77% | 75% | 73% | 86% | 73% | 63% | 63% | 67% | 69% |
| | Yₜₒₜₐₗ | 29% | 31% | 28% | 25% | 24% | 22% | 29% | 30% | 28% |
| | HPLC³ | 98.6% | 99.7% | 98.0% | 97.5% | 95.8% | 96.0% | 98.0% | 98.6% | 97.6% |
| | GC⁴ | 99.3% | 99.8% | 99.4% | 98.7% | 98.5% | 98.7% | 99.7% | 99.7% | 99.5% |

¹Crude yield combining EME and PEM
²Amount of EME calculated based on the GC peak area ratio of EME and PEM in isolated crude
³Sample preparation for HPLC purity assay: A 5 μL aliquot at a concentration of 10 mg/1.5 mL (methanol) was injected.
⁴Sample preparation for GC purity assay: An EME free base solution was prepared as follows: EME HCl (10 mg) was suspended in CH₂Cl₂ (2 mL) and aq. 0.05M Na₂CO₃ solution (0.8-1 mL) was added. The mixture was vigorously shaken for 20 sec. The organic layer was separated and the aqueous layer was back extracted with CH₂Cl₂ (2 mL). The combined organic layer was filtered through a pipette containing a cotton plug and anhydrous K₂CO₃. A 1 μL aliquot (7-10 mg/1 mL CH₂Cl₂) of the organic layer was injected.

Discussion of Comparative Examples

Low levels of impurities and high EME/PEM ratios were achieved for batches A1-A3 compared to B1-B3, as shown in Table 3. In batches A1-A3, the total impurities were <0.4%, EME/PEM ratios were from about 2.4/1 to about 3.1/1. In batches B1-B3, total impurities were from 0.2-4.9%, and EME/PEM ratios were from 1.6/1 to 2.6/1. Although almost none of the impurities were detected in batches C1-C3, only modest EME/PME ratios were achieved from 1.8/1 to 1.9/1.

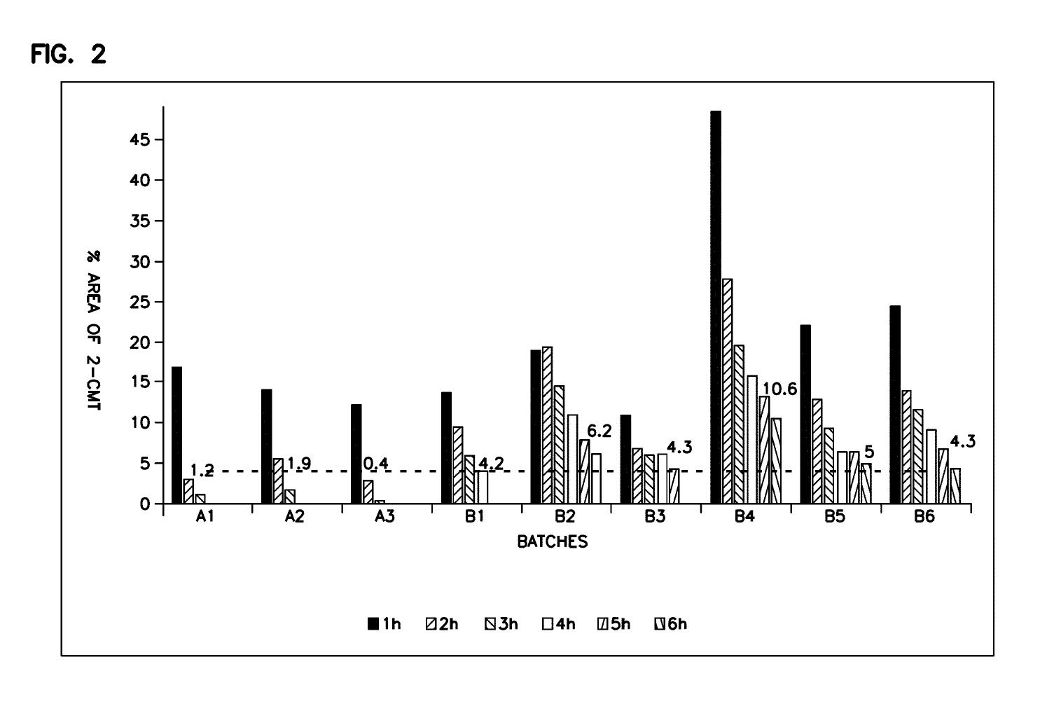

A comparison of reaction time for batches A1-A3, B1-B3 and C1-C3 is shown in FIG. 2. After 1 h, about 75-86% conversion of 2-CMT was achieved in batches A1-A3 and B1-B3. After 3 h, the amount of unreacted 2-CMT fell below 2% in batches A1-A3, whereas the overall rate of conversion of 2-CMT to EME/PEM was slow in batches B1-B3: 4.2, 6.2 and 4.3% remaining of 2-CMT after 4, 6 and 5 h, respectively (FIG. 2). The slow conversion for batches B1-B3 compared to batches A1-A3 might be associated with the high-water solubility of sodium formate that was produced as a by-product during the sodium amalgam reduction. Solubility of sodium formate and sodium sulfate by-products are shown in Table 4.

TABLE 4
Solubility of Na₂SO₄ and HCO₂ Na in water (100 mL)

| | | |
|---|---|---|
| Temp | Na₂SO₄ | HCO₂ Na |

| | | | | |
|---|---|---|---|---|
| 0° | C. | 4.9 | g | 43.9 g |
| 10° | C. | 9.1 | g | 62.5 g |
| 20° | C. | 19.5 | g | 81.2 g |

Without being bound by theory, the high-water solubility of sodium formate may lead to an increase in solution viscosity which tends to slow down the rate of conversion of residual 2-CMT at a mid-late stage of the reaction. A similar trend was observed for comparative batches C1-C3 to that observed in B1-B3, but the reaction rate was even slower. It may be possible that the overall rate of conversion was influenced by a lower reaction temperature (0-5° C. for C1-3 vs 5-10° C. for B1-B3, Table 1).

Due to the heterogeneous nature and formation of inorganic salts, the rate of sodium amalgam reduction tended to be slower at the mid-late stage. The reaction media in batches B1-B3 and C1-C3 exhibited high viscosity due to by-product formation of the highly water soluble sodium formate, resulting in a distinct negative effect on the reaction rate as compared to the reaction medium in A1-A3 containing precipitates (sodium sulfate). An increase of the effective collision frequency between two reactants (sodium amalgam and 2-CMT) is necessary to enhance the overall reaction rate.

In summary, the data shown in FIG. 2 illustrate distinct advantages exhibited by inventive Method A (A1-A3) compared to prior art comparative U.S. Pat. No. 7,855,296 Method of C (C1-C3). Although by-product sodium formate is water soluble, a slower rate of conversion of 2-CMT to EME/PEM and higher residual starting material were observed when using the comparative Method C with formic acid compared to Method A with sulfuric acid. Method C required greater than 6 h reaction time whereas Method A the reactions were complete in less than 3 h, despite the fact that the by-product sodium sulfate was allowed to remain as a precipitate throughout the reaction. In only 3 h reaction time, Method A with sulfuric acid resulted in an average of 98.8% conversion, or of over 98% 2-CMT conversion. In contrast, even after 6 h, comparative Method C resulted in an average of 93.4% conversion of 2-CMT by GC area %, as shown in FIG. 2 and Table 2.

Method B employed formic acid and was used to compare and contrast with the improved results exhibited in Method A which are due at least in large part to the use of sulfuric acid, and not solely other reaction conditions. Method B exhibited somewhat faster rate (4-6 h) than comparative Method C (6 h), but was slower than Method A (3 h). Method B exhibited an average of 95.1% conversion of 2-CMT, as shown in FIG. 2 and Table 2. Method B required increased amounts of formic acid and resulted in lower total yield (Table 3) and higher impurities 1 and 2 than comparative Method C (Table 2).

Basification Step and Extraction of EME Free Base

After reaction completion, the reaction mixture was basified with sodium carbonate to convert EME salts to free base. In batches A1-A3 only about 17 g of sodium carbonate was required to reach the pH 9-10. In contrast, 72-108 g of sodium carbonate was required to raise the pH of the mixture to 9-10 for batches B1-B3. Without being bound by theory, formation of a formic acid buffer which can resist the change of pH may cause this effect. In addition, a relatively large amount of carbon dioxide was produced in batches B1-B3 during basification and troublesome gas bubbles were also formed.

A relatively small amout of formic acid was consumed during the reaction in comparative batches C1-C3 compared to B1-B3 (˜107 mL vs ˜64 mL for B1-B3 and C1-C3, respectively) as the limits of pH increased (pH 4.5-5.5 in C1-C3 vs. pH 3.5-4.5 for B1-B3). Thus pH control and less laborous basification process was observed for C1-C3 compared to B1-B3. The difference in pH of the reaction mixture from 3.5-4.5 to 4.5-5.5 had little impact on overall reaction profiles. The basification process in C1-C3 was conducted with ammonium hydroxide (28-30%); it was convenient for use and required only 6-9 mL of ammonium hydroxide. Also, no gas bubbles were formed as opposed to the use of sodium carbonate. Additional study may be needed to evaluate the pros and cons of using ammonium hydroxide.

High crude yields were obtained in sodium amalgam reduction step for batches C1-C3 (66-80% vs 50-58% for C1-C3 and A1-B3, respectively, Table 3) that could be attributed to the use of large volume of extracting solvent (536 mL of CHCl₃ for C1-C3 vs 230 mL of CH₂Cl₂ for A1-B3, Tabel 1), or use of ammonium hydroxide may facilitate the extraction process.

Salting Step-Production of EME HCl from EME Base

Batches A1-A3 using sulfuric acid showed better overall yield and purity compared to batches B1-B3 using formic acid under the same reaction conditions (28-31% for A1-A3 vs 22-25% for B1-B3. The HPLC purity was also higher for A1-A3 (98.0-99.7%) than B1-B3, as shown in Table 5. In comparative batches C1-C3, HPLC purity of EME HCl was 97.6-98.6% with low impurities. A different procedure was used for HCl salt formation of EME free base. First, the crude EME free base was dissolved in CHCl₃, treated with HCl (2 M in ether) and subsequently, crude EME HCl salt was isolated. Then, the crude salt was further purified by tritulation with CHCl₃ to give the desired product with reasonably good purity.

After the reaction, the aqueous solution is removed from the reactor and possible traces of mercury are separated. In one embodiment, activated carbon is added to the aqueous solution and the mixture is stirred and then filtered to remove the carbon which absorbs any traces of mercury. Other methods of removing possible mercury contaminations from the aqueous solution are also possible.

In some embodiments, the reduction method comprises an extraction operation to extract the methylecgonine (EME) and pseudo-methylecgonine (PEM) from the filtered solution using methods known in the art to give pale yellow oil, which contains a mixture of methylecgonine and pseudo-methylecgonine.

In some embodiments, a method is provided for separating (−)-EME from a crude mixture of (−)-EME and PEM compromising stirring or triturating the mixture in cyclohexane, allowing the PEM to precipitate and filtering off the precipitated PEM.

In some embodiments, the methylecgonine (EME) is separated from the pseudo-methylecgonine (PEM) by HCl salt formation and selective crystallization.

In one embodiment, a method for forming EME HCl from a mixture of EME and PEM is provided comprising dissolving the mixture of EME and PEM in an alcoholic solvent and treating with HCl to form a reaction mixture; adding a counter solvent to the reaction mixture; and allowing the EME HCl to crystallize. In some embodiments, the alcoholic solvent is not ethanol. In some embodiments, the alcoholic solvent is isopropyl alcohol. In some embodiments, the counter solvent is acetone. In some embodiments, the HCl is methanolic HCl.

A method for separating EME from the PEM is provided comprising dissolving the mixture of EME and PEM in isopropyl alcohol and treating with methanolic HCl to form a reaction mixture. Following evaporation of solvent and trituration with fresh isopropyl alcohol, acetone is added and the EME HCl crystallizes upon standing at ambient temperature after about 16 h, as shown in Example 2.

In one embodiment, a method for forming EME HCl from a mixture of EME and PEM is provided comprising dissolving the mixture of EME and PEM in isopropyl alcohol and treating with methanolic HCl to form a reaction mixture; adding acetone to the reaction mixture; and allowing the EME HCl to crystallize. In some embodiments, the isopropyl alcohol in the reaction mixture is evaporated and replaced with fresh isopropyl alcohol before adding the acetone.

In some embodiments, the HCl solution of EME and PEM is held at a temperature of from 0-40° C., 10-35° C., 15-25° C. or ambient temperature to allow the EME HCl to precipitate.

In some embodiments, the HCl solution of EME and PEM is held at a temperature of from 0-40° C., 10-35° C., 15-25° C. to allow the EME HCl to precipitate over a period of 4-72 h, 6-48 h, or 12-20 h.

In another embodiment, the separation of EME from PEM may be conducted using two steps. In the first step of the separation operation, the oil is dissolved in a sufficient amount of an organic solvent, for example, cyclohexane. The pseudo-methylecgonine will partially precipitate out of the cyclohexane solution over time. In one embodiment, the cyclohexane solution is stirred or allowed to stand for 4-16 hours to allow sufficient time for the precipitation to occur. The precipitated pseudo-methylecgonine is separated from the cyclohexane mixture by filtration.

The filtrate is then evaporated to give pale yellow oil (which is a mixture of (−)-methylecgonine (EME) and pseudo-methylecgonine but which is substantially enriched with methylecgonine). Prior to evaporation, the filtrate may be stirred with silica gel, and filtered again to remove any impurities.

In the second part of the separation operation, the remaining pseudo-methylecgonine may be removed by methods known in the art. For example, separation is achieved by converting the methylecgonine (EME) and pseudo-methylecgonine to the corresponding hydrochlorides. Methylecgonine hydrochloride is practically insoluble in chloroform and precipitates, while pseudo-methylecgonine-HCl remains in solution. The precipitate may be removed by filtration and washed or otherwise purified to improve the purity of the methylecgonine hydrochloride (EME HCl). For example, in one embodiment, after filtering the formed solid is washed with chloroform twice and re-dissolved in a sufficient quantity of methanol, which is then evaporated to dryness. The solid residue is then stirred with a sufficient amount of chloroform, filtered again, washed twice with chloroform, washed twice again with hexane or some other volatile solvent to remove the chloroform and dried on air to give (−)-methylecgonine hydrochloride (EME HCl) as a snow-white solid, as described in Kuznetsov U.S. Pat. No. 7,855,296, which is incorporated herein by reference in its entirety.

Benzoylation of EME and HCl Salt Formation of Cocaine Hydrochloride

In some embodiments, the (−)-EME or salt thereof produced by a method as provided herein may be subjected to benzoylation by any method known in the art to produce cocaine.

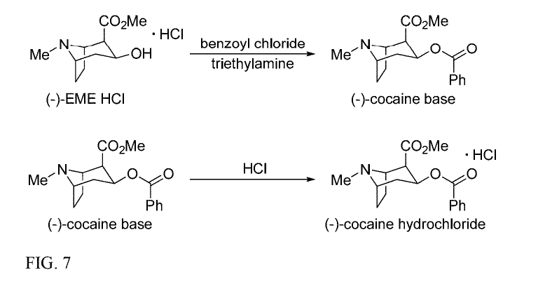

(−)-Cocaine or a pharmaceutically acceptable salt thereof may be produced from (−)-methylecgonine hydrochloride (EME HCl) by methods known in the art. FIG. 7 shows a scheme illustrating one embodiment for the benzoylation of (−)-methylecgonine hydrochloride into (−)-cocaine. The (−)-cocaine or pharmaceutically acceptable salt thereof created by this process can then be used as a component in the manufacture of other products.

In some embodiments, (−)-cocaine hydrochloride is produced by the method of DeJong 1940, Ishihara 1931, or Kuznetsov U.S. Pat. No. 7,855,296.

De Jong, Recueil des Travaux Chimiques des Pays-Bas, 1940, 59 (1), 27-30, discloses complete conversion is obtained in 10 hours by boiling an anhydrous benzene solution of l-ecgonine methyl ester (also known as (−)-methylecgonine, or EME) with benzoyl chloride (BzCl) in the presence of dry sodium carbonate, calcium oxide or a mixture of calcium oxide and hydroxide in chloroform or ether. In chloroform solution about 20 hours are necessary and in ethereal solution about 40 hours, when a mixture of calcium oxide and hydroxide is used.

Ishihara, K., Chem Abstracts 1931, 25, 4359, reports reaction of ecgonine methyl ester hydrochloride with BzCl in the presence of a phenol as catalyst with heating to 90° C. for 4 h, adding water and CHCl₃ to precipitate cocaine. Alternatively, Ishihara 1931 reports mixing ecgonine methyl ester hydrochloride and benzoyl chloride and heating in a closed vessel at 900 for 5 hours at a pressure of 300 lb. The reaction mixture is poured into water and extracted with CHCl₃, and cocaine is precipitated by adding alkali to the aqueous solution.

Kuznetsov U.S. Pat. No. 7,855,296 prepares (−)-cocaine by benzoylation of methylecgonine in chloroform with benzoyl chloride and triethylamine. Crude cocaine base was dissolved in tert-butyl methyl ether and treated with heptane to crystallize (−)-cocaine base.

FIG. 7 shows exemplary methods for converting (−)-EME to (−) cocaine base and subsequent hydrochloride salt formation to provide (−) cocaine hydrochloride.

In some embodiments, a method is provided for benzoylating ecgonine methyl ester or a salt thereof by mixing with benzyl chloride and a base. In some embodiments, the base is selected from trimethylamine, sodium carbonate, calcium oxide, or calcium hydroxide to form (−)-cocaine base. The cocaine base may be crystallized by any method known in the art. For example, the crude cocaine base may be dissolved in tert-butyl methyl ether and precipitated by addition of heptane by the method of Kuznetsov U.S. Pat. No. 7,855,296.

In some embodiments, cocaine hydrochloride may be formed from (−)-cocaine base by any method known in the art.

Methods for evaluation of impurities and residual solvents for synthetically-derived cocaine hydrochloride prepared according to the present disclosure and comparative naturally-derived cocaine hydrochloride USP (Mallinckrodt Pharmaceuticals) are provided in examples 6A-D and 7. In embodiments, the disclosure provides isolated (−)-cocaine hydrochloride having not more than 0.15%, not more than 0.1%, or not more than 0.05% benzoic acid by HPLC, as shown in Table 9.

In embodiments, the disclosure provides isolated cocaine hydrochloride having not more than 0.5%, not more than 0.1%, or not more than 0.07% benzoyl ecgonine by HPLC, as shown in Table 9.

In embodiments, isolated cocaine hydrochloride is provided having not more than 0.5%, not more than 0.3%, or not more than 0.2% of Total Impurities by HPLC, as shown in Table 9.

In embodiments, isolated cocaine hydrochloride is provided having not more than 50 ppm ethanol, not more than 25 ppm ethanol, or not more than 10 ppm ethanol when tested according to USP protocols for cocaine hydrochloride.

In some embodiments, isolated cocaine hydrochloride is provided that is isolated synthetic cocaine hydrochloride.

Compositions

In some embodiments, compositions are provided comprising the isolated cocaine hydrochloride prepared by a method of the disclosure. In some embodiments, a composition is provided comprising (−)-cocaine hydrochloride having no more than 100 ppm ethyl cocaine and a pharmaceutically acceptable carrier.

In some embodiments the disclosure provides a pharmaceutical composition comprising a pharmaceutically effective amount of cocaine hydrochloride having not more than 0.15% (1500 ppm), 0.1% (1000 ppm), 0.05% (500 ppm), 0.025% (250 ppm), 0.01% (100 ppm), 0.005% (50 ppm), 0.0025% (25 ppm), 0.001% (10 ppm), 0.0005% (5 ppm), 0.0001% (1 ppm) of an impurity selected from the group consisting of ethyl cocaine, 2′-furanoylecgonine methyl ester (FEME), ecgonine, (−)-ecgonine methyl ester, pseudococaine, dehydrococaine, benzoylpseudotropine, 2,3-dehydrobenzoyltropine (also known as dehydrobenzoyl pseudotropine), and a pharmaceutically acceptable carrier.

According to another aspect, the present invention provides a pharmaceutical composition, which comprises a therapeutically-effective amount of one or more compounds of the present invention or a pharmaceutically-acceptable salt, ester or prodrug thereof, together with a pharmaceutically-acceptable diluent or carrier.

Pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose, microcrystalline cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) lubricants, such as magnesium stearate, calcium stearate, zinc stearate, sorbitan monostearate, sucrose monopalmitate, glycerol dibehenate, and stearic acid; (16) alginic acid; (17) pyrogen-free sterile water; (18) isotonic saline; (19) Ringer's solution; (20) ethyl alcohol; (21) phosphate buffer solutions; (22) aqueous solution of citric acid or a hydrate thereof; (23) polymers and time release agents; (24) bioavailability enhancers and bioavailability controllers/inhibitors; (25) preservatives; and (26) other non-toxic compatible substances employed in pharmaceutical formulations.

Other non-toxic compatible substances include optional flavorings and/or sweeteners.

In another embodiment, compositions of the disclosure can optionally further comprise one or more flavoring agents. The optional flavoring agent is added to increase patient acceptability and compliance with the recommended dosing schedule. The flavoring agents that may be used include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixture. Commonly used flavors include mints such as peppermint, menthol, artificial vanilla, cinnamon derivatives, and various fruit flavors, whether employed individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. In a specific aspect, the flavoring is selected from a cherry or orange flavoring.

Various sweeteners can be optionally used in the solution, tablet, liquid, capsule, lozenge or troche formulations of the disclosure. Examples of carbohydrates and sweeteners include monosaccharides such as glucose and fructose, disaccharides such as maltose, sucrose, other ordinary sugars, sugar alcohols such as xylitol, sorbitol, glycerin and erythritol, polysaccharides such as dextrin and cyclodextrin, and oligosaccharides such as fructo-oligosaccharide, galacto-oligosaccharide and lacto-sucrose. Other sweeteners include natural sweeteners such as thaumatin, stevia extract, Luo Han Guo (Lo Han fruit), rebaudioside A, glycyrrhizinic acid, etc. and synthetic sweeteners such as saccharin, aspartame, azesulfame potassium, etc.

Optionally various FD& C dyes or opacifiers can be employed in the compositions. In various aspects, the FD&C dye is selected from one or more of FD&C Red No. 3, Red No. 40, Red No. 33, Yellow No. 6, Yellow No. 6 lake, Yellow No. 5 lake, Yellow No. 5, Green No. 3, Blue No. 1 and Blue No. 2, and D&C Yellow No. 10. In one specific aspect, a composition is provided comprising D&C Yellow No. 10, and FD&C green No. 3. In some embodiments, the pharmaceutical composition may include from 0.001-0.05, or 0.002-0.01 mg/mL of one or more dyes.

Preservatives can be included in the pharmaceutical compositions and may be selected from any preservative known in the art, or a combination thereof. In some embodiments, one or more preservatives may include methyl parabens, ethyl parabens, propyl parabens and combinations, sodium benzoate, benzoic acid, sorbic acid, potassium sorbate, propionic acid, methyl paraben/sodium benzoate combination. In a specific embodiment, the preservative is sodium benzoate. In some embodiments, the pharmaceutical composition may include from 0.001-2.0, 0.01-1.5, 0.05-1.0 mg/mL of one or more preservatives.

The compositions may be formulated for any route of administration, in particular for topical, oral, rectal, transdermal, or intranasal administration. In a specific embodiment, compositions are provided for introduction of local (topical) anesthesia of accessible mucous membranes of the oral, laryngeal and nasal cavities in a subject, comprising administering a composition comprising cocaine hydrochloride having no more than 10 ppm ethyl cocaine, and a pharmaceutically acceptable carrier.

The compositions may be formulated in any conventional form, for example, as topical solution, dispersible tablets, diskets dispersible tablets, suspensions, dispersions, troche, syrups, sprays, gels, suppositories, and emulsions. In specific embodiments, the composition is in the form of a topical solution.

As is well known in the medical arts, dosages for any one subject may depend upon many factors, including the patient's size, body surface area, age, the particular compound to be administered, sex, time and route of administration, general health, and interaction with other drugs being concurrently administered. Depending on the target sought to be altered by treatment, pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include topical or transmucosal administration; as well intranasal administration. In some embodiments, dosage forms for transmucosal administration include, but are not limited to aqueous solution, fast melt, buccal or sublingual dosage forms.

Pharmaceutical compositions suitable for use in the present application include compositions wherein the active ingredients (e.g., cocaine, cocaine hydrochloride, and combinations thereof), comprising not more than 0.15%, 0.10%, 0.05%, 500 ppm, 100 ppm, 50 ppm, 10 ppm, 5 ppm, or 1 ppm ethyl cocaine, not more than 0.5% ecgonine, not more than 1.5% (−)-ecgonine methyl ester, and not more than 6.5% benzoyl Ecgonine. In some embodiments, the active ingredient includes not more than 0.2% of pseudococaine, dehydrococaine, benzoylpseudotropine, or 2,3-dehydrobenzoyltropine. For example, in a preferred embodiment, an effective amount of a topical pharmaceutical composition comprises an amount of cocaine hydrochloride comprising not more than 100 ppm ethyl cocaine. Determination of effective amounts is well within the capability of those skilled in the art, especially in light of the disclosure provided herein.

Pharmaceutical compositions suitable for use in the present application include compositions wherein the active ingredients (e.g., cocaine, cocaine hydrochloride, and combinations thereof), comprising not more than 0.15%, 0.1%, 500 ppm, 100 ppm, 50 ppm, 10 ppm, or 1 ppm of ethyl cocaine, not more than 0.5% ecgonine, not more than 1.5% (−)-ecgonine methyl ester, not more than 6.5% benzoyl Ecgonine, and not more than 0.2% of pseudococaine, dehydrococaine, benzoylpseudotropine, or 2,3-dehydrobenzoyltropine, is contained in an effective amount to achieve the intended purpose.

In one embodiment, a cocaine hydrochloride composition is provided that is a topical aqueous composition comprising an effective amount of cocaine hydrochloride having not more than 100 ppm ethyl cocaine, citric acid, and sodium benzoate in water. In some aspects, the composition further contains one or more dyes. In a specific embodiment, an aqueous pharmaceutical composition is provided comprising 4% (40 mg/mL) or 10% (100 mg/mL) of ethyl cocaine free cocaine hydrochloride, citric acid, sodium benzoate, water, D&C Yellow No. 10, and FD&C Green No. 3. In another specific embodiment, an aqueous pharmaceutical composition is provided comprising 4% (40 mg/mL) or 10% (100 mg/mL) of ethylcocaine free cocaine hydrochloride, citric acid anhydrous, sodium benzoate, water, D&C Yellow No. 10, and FD&C Green No. 3. In some aspects, the composition further comprises one or more flavorings.

In another specific embodiment, a cocaine hydrochloride composition is provided that is a topical solution comprising an effective amount of cocaine hydrochloride having not more than 100 ppm ethyl cocaine, citric acid, purified water, and sodium benzoate. In a specific embodiment, a composition is provided that is an topical solution comprising 100 mg/mL cocaine hydrochloride having not more than 100 ppm ethyl cocaine, citric acid, purified water, and sodium benzoate. In a specific embodiment, an aqueous pharmaceutical composition is provided comprising 10% or 100 mg/mL cocaine hydrochloride having not more than 100 ppm ethyl cocaine, citric acid, sodium benzoate, water, D&C Yellow No. 10, and FD&C Green No. 3.

In some specific embodiments, an effective amount of cocaine hydrochloride having not more than 100 ppm ethyl cocaine in a topical composition is selected from about 10 mg, 20 mg, 30 mg, 40 mg, 50 mg, 60 mg, 70 mg, 80 mg, 90 mg, 100 mg, 110 mg, 120 mg, 130 mg, 140, mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg or any dose in between. In some embodiments, an effective amount of cocaine hydrochloride having not more than 100 ppm ethyl cocaine in a topical composition is selected from 0.1-3 mg/kg, 0.5-2.5 mg/kg, or 1-2 mg/kg.

Administration

In some embodiments, methods are provided for introduction of local (topical) anesthesia of accessible mucous membranes of the oral, laryngeal and nasal cavities in a subject in need thereof, comprising administering a composition comprising cocaine hydrochloride and a pharmaceutically acceptable carrier, wherein the cocaine hydrochloride has less than 100 ppm, less than 50 ppm, less than 20 ppm, or less than 10 ppm ethyl cocaine. The composition may be administered by means of an absorbent application, such as a cotton applicator, pledget, or pack, instilled into a cavity, or as a spray.

In some embodiments the disclosure provides a method of treating a subject in need thereof, comprising administering a composition comprising an effective amount of a pharmaceutical composition comprising a pharmaceutically effective amount of (−)-cocaine hydrochloride having not more than 0.15%, 0.1%, 0.05% (500 ppm), 0.025% (250 ppm), 0.01% (100 ppm), 0.005% (50 ppm), 0.0025% (25 ppm), or 0.001% (10 ppm) of ethyl cocaine, not more than 0.5%, 0.3%, 0.1% ecgonine, not more than 1.5%, 1.0%, 0.5%, 0.15% (−)-ecgonine methyl ester, not more than 6.5%, 5%, 1%, 0.5%, 0.15% benzoyl ecgonine, not more than 0.2% of an impurity selected from the group consisting of pseudococaine, dehydrococaine, benzoylpseudotropine, FEME, and 2,3-dehydrobenzoyltropine, and a pharmaceutically acceptable carrier.

In some embodiments the disclosure provides a method of treating a subject in need thereof, comprising administering a pharmaceutical composition comprising an effective amount of cocaine hydrochloride having not more than 0.15%, 0.1%, 500 ppm, or 100 ppm of ethyl cocaine.

Indications for cocaine hydrochloride compositions provided herein include use as a local anesthetic agent. Cocaine hydrochloride compositions are provided for topical administration to produce local anesthesia of accessible mucous membranes or oral, laryngeal, and nasal cavities. Compositions are indicated for the introduction of local (topical) anesthesia for diagnostic procedures and surgeries on or through the accessible mucous membranes of the nasal cavities.

The dosage depends upon the area to be anesthetized, vascularity of the tissues, individual tolerance, and the technique of anesthesia. In some embodiments, the introduction of local anesthesia may be diagnostic surgery, rhinoplasty, endoscopy, and bronchoscopy.

In some embodiments, the effective amount of cocaine hydrochloride is selected from an amount of from 10 mg to 400 mg, 20 mg to 300 mg, or 40 mg to 150 mg cocaine hydrochloride having not more than 0.15%, 0.10%, 0.05%, or not more than 100 ppm ethyl cocaine.

In some embodiments, an effective amount of cocaine hydrochloride is selected from an amount within a range of about 10-400 mg, 20-300 mg, 30-250 mg, 40-200 mg, or 50-100 mg per dose. In some specific embodiments, the effective amount is selected from 10 mg, 20 mg, 30 mg, 40 mg, 50 mg, 60 mg, 70 mg, 80 mg, 90 mg, 100 mg, 125 mg, 140 mg, 150 mg, 175 mg, 200 mg, 225 mg, 250 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, per dose, or any dose in between. In some embodiments, the cocaine hydrochloride is present in 10, 20, 30, 40, 50, 60, 70, 80, 90, 100, 110, 120, 130, 140, 150, 160, 170, 180, 190, or 200 mg/mL in the composition. In some specific embodiments, the effective amount of the cocaine hydrochloride is present at a concentration selected from 40 mg/mL, or 100 mg/mL, in the composition.

In some embodiments, the cocaine hydrochloride composition may be a solution composition that is topically applied by soaking a pledget, sponge, strip, patty, sponge, applicator, or ball made from rayon, cotton, or cellulose fiber, in the solution and topically applying to a mucous membrane, for example within the nasal cavity for a period of 1, 2, 5, 10, 15, 20, 25, 30, 35, 40, or 45 minutes, or any period of time in between. The application may be a single application, or may be repeated for a total of one, two or three applications for example, using multiple pledgets or some other applicator, depending on the procedure.

In some embodiments, the cocaine hydrochloride composition is administered one per day (q.d.), twice per day (b.i.d.), three times per day (t.i.d.), four times per day (q.i.d.), or more. In some embodiments, the composition is for administration in an as needed basis.

The dosage depends upon the area to be anesthetized, vascularity of the tissues, individual tolerance, and the technique of anesthesia. In some embodiments, the introduction of local anesthesia may be diagnostic surgery, rhinoplasty, endoscopy, and bronchoscopy.

### EXAMPLES

In the examples below, temperatures are provided in degrees Celsius and all parts and percentages are by weight, unless otherwise specified. Reagents may be purchased from commercial suppliers, such as Sigma-Aldrich Chemical Company, and may be used without further purification unless otherwise indicated. Reagents may also be prepared following standard literature procedures known to those skilled in the art. Solvents may be purchased from commercial suppliers, or may be purified using standard methods known to those skilled in the art, unless otherwise indicated.

The compound structures in the examples below were confirmed by one or more of the following methods: proton magnetic resonance spectroscopy, mass spectroscopy, and melting point. Proton magnetic resonance (¹H NMR) spectra were determined using an NMR spectrometer operating at 300 MHz field strength. Chemical shifts are reported in the form of delta (δ) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz). Mass spectra (MS) data are obtained using a mass spectrometer with MALDI-TOF, APCI or ESI ionization.

### Example 1. Continuous Reduction of 2-CMT to Form EME

This example shows three methods for reducing 2-CMT bitartrate using electrochemically generated sodium amalgam (FIG. 1) and an acid. Example 1A shows a representative test procedure A where the acid is sulfuric acid. Example 1B shows a different representative test procedure B where the acid is formic acid. Example 1C shows a comparative procedure C where the acid is formic acid. Comparative procedure C was performed according to the method of U.S. Pat. No. 7,855,296. In each method, the sodium amalgam is continuously supplied from an electrolyzing unit to a reactor containing the aqueous solution of (+)-2-carbomethoxytropinone bitartrate and the acid. The spent amalgam is continuously removed from the reactor and transferred to the electrolyzing unit for regeneration.

### Example 1A: Representative Procedure for Experimental Group A: Batch A1

A three-necked 500 mL jacket reactor was equipped with a mechanical stirrer, a digital thermometer, a pH probe and a graduated addition funnel. The reactor was connected to an electrolytic cell via the bottom drain. The cell contained approximately 4.3 kg of mercury which was covered by a 600 mL of 50 wt % NaOH solution. The nickel anode was placed in the solution and a constant current (4.5 A, 7-12 V) electrolysis was carried out for ˜3 h to provide formation of sodium amalgam which was pumped by a peristaltic pump to the top inlet of the jacketed reactor and allowed to flow back through the bottom drain to the electrolytic cell.

On the other hand, a 500 mL round bottom flask was charged with water (130 mL) and (+)-2-CMT bitartrate (Item #21-157, Batch #140079, manufactured by Strides Shasun Limited) (30.56 g, 88.00 mmol) was added portionwise. The pH of the resulting suspension was ˜3.21 which was then brought to pH 4.7 with aqueous 50% NaOH (4 mL). The resulting mixture was stirred for >30 min to ensure complete dissolution of 2-CMT. Activated carbon (3.36 g) was then added to the solution. After stirring for 5 min, the activated carbon was filtered off and washed with water (25 mL×2). The combined solutions in an Erlenmeyer flask were cooled to 5° C. and transferred into the above three-necked 500 mL jacket reactor while the peristaltic pump was stopped temporarily. The flask was rinsed with water (10 mL).

Direct electric current (4.5 A, 7-12 V) was passed through the electrolytic cell containing nickel anode and copper/mercury cathode. Sodium amalgam formed in the electrolysis was continuously circulated to the jacketed reactor via a peristaltic pump as described before. The temperature of the reaction mixture was maintained at 5-10° C. throughout the reduction process. The pH of the reaction mixture was monitored and continuously adjusted to 3.5-4.5 by adding 40% H₂SO₄. The progress of the reaction was monitored by GC. After ˜1 h, a white solid (sodium sulfate) began to precipitate. After 3 h 1.2% 2-CMT remained and the reaction was stopped. The total volume of 40% H₂SO₄ consumed during the reaction was 140 mL.

After the reaction was stopped, water (108 mL) was charged into the reactor. The temperature was then raised to 25° C. and the mixture was stirred for 20 min to ensure that sodium sulfate formed during the reaction was fully dissolved. The resulting mixture was transferred into a 2 L Erlenmeyer flask and the reactor was rinsed with water (108 mL×2). The combined mixtures were filtered through a filter paper to remove a trace of mercury and washed with water (108 mL). The combined aqueous filtrates were basified with sodium carbonate. A total of 17 g of the base was added to bring the pH to 9.2. The product portion was extracted with dichloromethane (80 mL×1, then 50 mL×3); GC analysis, 2-CMT/EME/PEM=2.3/71.9/25.8. The combined extracts were treated with silica gel (4.9 g), stirred for 5 min, filtered, washed with CH₂Cl₂ (30 mL), and concentrated in vacuo. The crude product mixture containing ecgonine methyl ester (EME) and pseudoecgonine methyl ester (PEM) was dissolved in cyclohexane (60 mL) and concentrated in vacuo. This solvent swap procedure was repeated three times to afford the crude mixture (9.08 g). The crude was dissolved in cyclohexane (130 mL) and stirred overnight at around 18° C. The precipitate (PEM) was filtered, washed with cyclohexane (30 mL) and air dried to give PEM (775 mg). The combined filtrates were mixed with MeOH (50 mL), treated with conc-HCl (3.3 mL) at 5-10° C. and stirred vigorously at around 20° C. for 10-30 min. The bottom layer (pH=2-3) consisting of aqueous methanol was separated and the upper layer (cyclohexane) was back extracted with MeOH (20 mL) and water (2.4 mL). The combined extracts were concentrated in vacuo and the residue was treated with 2-propanol (20 mL) and acetone (86 mL). The mixture was stirred for 0.5-1 h at around 15° C., filtered, washed with 2-propanol (7.5 mL) and acetone (15 mL), and dried in air to give EME HCl (6.04 g, 29%). HPLC purity by Method A, 98.6% (t_{R}=9.74 min); GC purity, 99.3% (t_{R}=10.95 min). Further analytical data is shown in Table 5.

### Example 1B: Representative Procedure for Experimental Group B: Batch B1

The three-necked 500 mL jacket reactor system and electrolysis conditions were identical to that of batch A1.

A 500 mL round bottom flask was charged with water (130 mL) and (+)-2-CMT bitartrate (30.56 g, 88.00 mmol) was added portionwise. The pH of the resulting suspension was ˜3.14 which was then brought to pH 4.7 with aqueous 50% NaOH (4 mL). The resulting mixture was stirred for >30 min to ensure complete dissolution of 2-CMT. Activated carbon (3.36 g) was then added to the solution. After stirring for 5 min, the activated carbon was filtered off and washed with water (25 mL×2). The combined solutions in an Erlenmeyer flask were cooled to 5° C. and transferred into the above three-necked 500 mL jacket reactor while the peristaltic pump was stopped temporarily. The flask was rinsed with water (10 mL).

Direct electric current (4.5 A, 7-12 V) was passed through the electrolytic cell containing nickel anode and copper/mercury cathode. Sodium amalgam formed in the electrolysis was continuously circulated to the jacket reactor via a peristaltic pump as described before. The temperature of the reaction mixture was maintained at 5-10° C. throughout the reduction process. The pH of the reaction mixture was monitored and continuously adjusted to 3.5-4.5 by adding formic acid. The progress of the reaction was monitored by GC. After 4 h, 4.2% 2-CMT remained and the reaction was stopped. The total volume of formic acid consumed during the reaction was 92 mL.

Water (108 mL) was charged into the reactor and the temperature was then raised to 25° C. After the stirring for 20 min, the mixture was transferred into a 2 L Erlenmeyer flask and the reactor was rinsed with water (108 mL×2). The resulting mixtures were filtered through a filter paper to remove a trace of mercury and washed with water (108 mL). The combined aqueous filtrates were then basified with sodium carbonate. A total of 74 g of base was added to bring the pH to 9.2. The product portion was extracted with dichloromethane (80 mL×1, then 50 mL×3); GC analysis, 2-CMT/EME/PEM/impurity 1=4.2/55.7/35.2/4.9. The combined extracts were treated with silica gel (4.9 g), stirred for 5 min, filtered, washed with CH₂Cl₂ (30 mL), and concentrated in vacuo. The crude product mixture containing ecgonine methyl ester (EME) and pseudoecgonine methyl ester (PEM) was dissolved in cyclohexane (60 mL) and concentrated in vacuo. This solvent swap procedure was repeated three times to afford the crude mixture (9.18 g). The crude was dissolved in cyclohexane (130 mL) and stirred overnight at around 18 OC. The precipitate (PEM) was filtered, washed with cyclohexane (30 mL) and air dried to give PEM (1.09 g). The combined filtrates were mixed with MeOH (50 mL), treated with conc-HCl (3.3 mL) at 5-10° C. and stirred vigorously at around 20° C. for 10-30 min. The bottom layer (pH=2-3) consisting of aqueous methanol was separated and the upper layer (cyclohexane) was back extracted with MeOH (20 mL) and water (2.4 mL). The combined extracts were concentrated in vacuo and the residue was treated with 2-propanol (20 mL) and acetone (86 mL). The mixture was stirred for 0.5-1 h at around 15° C., filtered, washed with 2-propanol (7.5 mL) and acetone (15 mL), and dried in air to give EME HCl (5.20 g, 25%). HPLC purity by Method A, 97.5% (t_{R}=9.69 min); GC purity, 99.7% (t_{R}=10.91 min). Further analytical data is shown in Table 5.

### Example 1C: Representative Procedure for Experimental Group C: Batch C2

The three-necked 500 mL jacket reactor system and electrolysis conditions were identical to that of batch A1.

A 500 mL round bottom flask was charged with water (134 mL) and (+)-2-CMT bitartrate (30.56 g, 88.00 mmol) was added portionwise. The pH of the resulting suspension was ˜3.35 which was then brought to pH 5.7 with aqueous 50% NaOH (5 mL). The resulting mixture was stirred for >30 min to ensure complete dissolution of 2-CMT. After cooling to 5° C., the solution was transferred into the above three-necked 500 mL jacket reactor while the peristaltic pump was stopped temporarily. The flask was rinsed with water (10 mL).

Direct electric current (4.5 A, 7-12 V) was passed through the electrolytic cell containing nickel anode and copper/mercury cathode. Sodium amalgam formed in the electrolysis was continuously circulated to the jacket reactor via a peristaltic pump as described before. The temperature of the reaction mixture was maintained at 0-5° C. throughout the reduction process. The pH of the reaction mixture was monitored and continuously adjusted to 5.4-5.9 by adding formic acid. The progress of the reaction was monitored by GC. After 6 h, 5.0% 2-CMT remained and the reaction was stopped. The total volume of formic acid consumed during the reaction was 67 mL.

Water (108 mL) was charged into the reactor and the temperature was then raised to 25° C. After stirring for 20 min, the mixture was then transferred into a 2 L Erlenmeyer flask and the reactor was rinsed with water (108 mL×2). The resulting mixtures were filtered through a filter paper to remove a trace of mercury and washed with water (25 mL×2). Activated carbon (3.36 g) was then added to the solution. After stirring for 5 min, the activated carbon was filtered off and washed with water (108 mL). The combined aqueous filtrates were then basified with ammonium hydroxide solution (28-30%). A total of 7 mL of base was added to bring the pH to 9.5. The product portion was extracted with chloroform (134 mL×4); GC analysis, 2-CMT/EME/PEM=5.5/61.5/33.3. The combined extracts were dried with sodium carbonate (3.26 g), stirred for 5 min, filtered and concentrated in vacuo. The crude product mixture containing ecgonine methyl ester (EME) and pseudoecgonine methyl ester (PEM) was dissolved in cyclohexane (60 mL) and concentrated in vacuo. This solvent swap procedure was repeated two times to afford the crude mixture (12.73 g). The crude was dissolved in cyclohexane (122 mL) and stirred overnight at around 18 OC. The precipitate (PEM) was filtered, washed with cyclohexane (30 mL) and air dried to give PEM (2.47 g). The combined filtrates was treated with silica gel (4.9 g), stirred for 5 min, filtered, washed with cyclohexane (30 mL), and concentrated in vacuo (7.84 g). Then, a solvent swap to CHCl₃ was performed; the crude was dissolved in CHCl₃ (20 mL) and concentrated in vacuo. The resulting crude product was dissolved in CHCl₃ (51 mL) and treated with 2 M HCl in ether (21.5 mL); 1.05-1.1 equivalent of 2 M HCl in ether was added. After stirring vigorously at 20° C. for >30 min, the mixture was filtered and washed with CHCl₃ (26 mL×2). Crude EME HCl was re-dissolved in MeOH (51 mL) and concentrated in vacuo. The solid residue was stirred in CHCl₃ (34 mL) for 30 min, filtered, washed with CHCl₃ (26 mL) and hexane (26 mL), and air-dried to give EME HCl (6.16 g, 30%). HPLC purity by Method A, 98.6% (t_{R}=8.98 min); GC purity, 99.7% (t_{R}=10.91 min). Further analytical data is shown in Table 5.

A summary of analyses for each batch of EME HCl produced in test Examples 1A and 1B and comparative Example 1C is shown in Table 5. HPLC was performed by Method A.

TABLE 5
Summary of Analytical Data for (—)-EME HCl

| Analysis | A1 | A2 | A3 | ^{d}A4 | B1 | B2 | B3 | C1 | C2 | C3 |
|---|---|---|---|---|---|---|---|---|---|---|
| HPLC | 98.6% | 99.7% | 98.0% | 99.6% | 97.5% | 95.8% | 96.0% | 98.0% | 98.6% | 97.6% |
| GC | 99.3% | 99.8% | 99.4% | ~100% | 98.7% | 98.5% | 98.7% | 99.7% | 99.7% | 99.5% |
| ^{a}[α]_{D}²⁵ | −49.0 | −49.3 | −51.2 | −50.5 | −47.7 | −47.9 | −48.0 | −50.8 | −51.3 | −50.7 |
| ^{b}m.p. (° C.) | 212.5 | 217.6 | 212.5 | 218.0 | 210.5 | 210.6 | 207.6 | 208.2 | 207.9 | 206.6 |
| ^{c}loss of | — | 6% | — | 3% | — | 7.05% | — | 0% | — | — |
| water | | (81.8-177.7) | | (52.5-150.3) | | (69.3-178.2) | | (50-150) | | |
| (° C.) | | | | | | | | | | |

^{a}(c 1, MeOH)
^{b}Measured by differential scanning calorimetry (DSC)
^{c}Weight-loss percentage due to the loss of solvent (water) which was measured by thermogravimetric analysis (TGA) at the indicated temperature range.
^{d}Methanolic HCl solution and isopropanol were used instead of conc. HCl in the salting step (see Experimental Section).

Literature values for (+)-ecgonine methylester hydrochloride ((+)-EME HCl):

[α]_{D}²⁴+52.3 (c 1, MeOH); m.p. 213-214° C. (Forensic Sci. Int., 1987, 33, 275 Casale, J. F.)

[α]_{D}²⁴+52.3 (c 1, MeOH); m.p. 213.5-214.5° C. (J. Heterocyclic Chem. 1987, 24, 19 Lewin, A. H. et al.).

### Example 2. Isolation of EME.HCl Via Salting with Methanolic HCl (3.0 M)

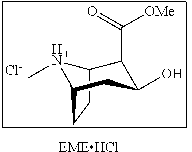

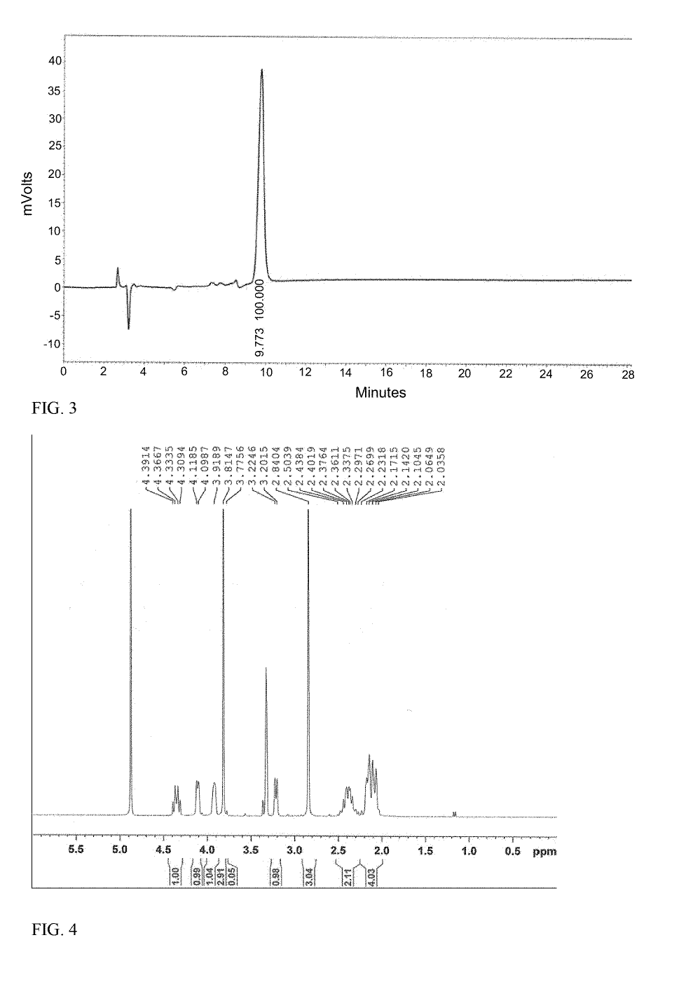

A crude mixture of EME and PEM (13.88 g, EME content 87.4% by GC, 70 mmol) was dissolved in 60 mL IPA and treated dropwise with 3.0 M HCl in MeOH (60 mL, 180 mmol, 2.57 eq relative to EME and PEM). The resulting mixture was stirred for 90 min at rt and 15 min at 45° C. before being concentrated on a rotary evaporator at 45-50° C. The residual was co-evaporated with IPA (40 mL×2) at 50-55° C. to give the crude EME HCl salt (wet weight 17.9 g). The crude product was triturated with 40 mL IPA at 50-55° C. for 15 min. Acetone (120 mL) was then added and the resulting mixture stirred at 55° C. for 25 min. After cooling to rt and stirred for 18 h, the precipitate was filtered and washed with a mixture of IPA (5 mL) and acetone (15 mL) and then with acetone (20 mL×2) to give 10.25 g EME HCl as white crystalline powders after drying in the air (71% yield based on EME base in the crude mixture). HPLC of the purified EME HCl was performed by Method A (FIG. 3). ¹H NMR (300 MHz, MeOH-d₄): δ 4.35 (dt, J=10.0 and 7.3 Hz, 8H), 4.11 (d, J=6.1 Hz, 1H), 3.92 (m, 1H), 3.81 (s, 3H), 3.21 (d, J=6.9 Hz), 2.84 (s, 3H), 2.27-2.50 (m, 2H), 2.04-2.23 (m, 4H), as shown in FIG. 4.

### Example 3. Isolation of EME.HCl Via Salting with Methanolic HCl (3.0 M)

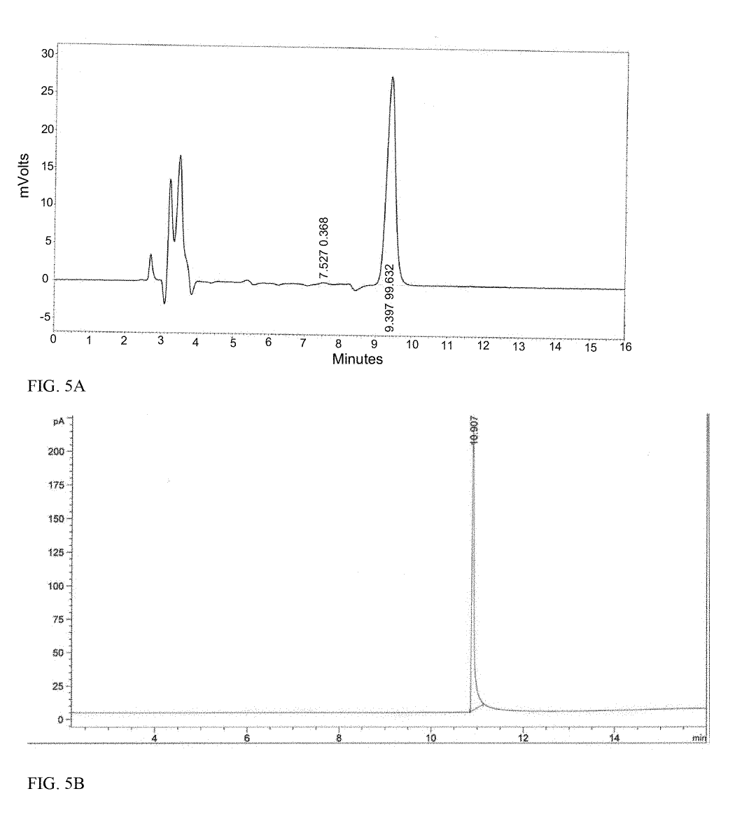

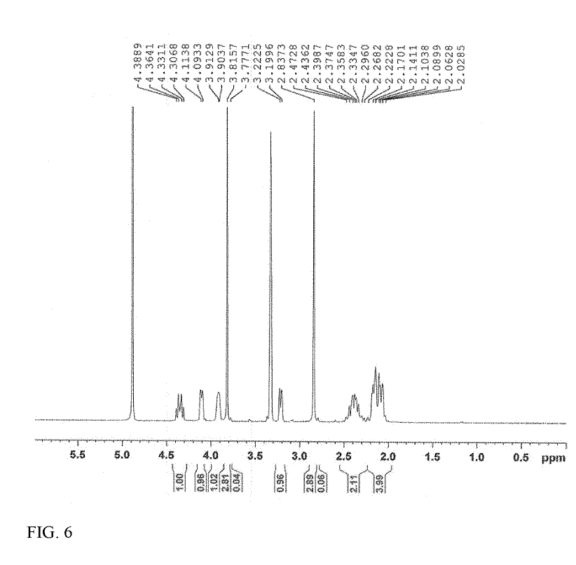

A crude mixture of EME and PEM (3.82 g, EME content 75.6% by GC, 14.5 mmol) was dissolved in 20 mL IPA and treated dropwise with 13 mL 3.0 M HCl in methanol (39 mmol, 2.7 eq). After stirred at rt for 60 min and then at 45° C. for 15 min, the solvent was removed on a rotavapor at 45-50° C. The residual was co-evaporated with IPA (10 mL×2) at 55° C. The solid EME HCl crude was taken up with 25 mL IPA and stirred at 55° C. for 15 min. Acetone (75 mL) was then added and the resulting mixture stirred at 60° C. for a gentle reflux for 30 min. After cooling to rt and stirred for 3 h, the precipitate was filtered and washed with a mixture of IPA (3 mL) and acetone (9 mL) and then with acetone (10 mL×2) to give 2.72 g EME HCl. (79.5% based on EME base in the crude mixture). HPLC of the EME HCl was performed by Method A showing a single peak eluting at 9.397 min retention time at 210 nm (99.63 area %), as shown in FIG. 5A. Evaluation of EME HCl produced by this method showed GC single peak at 10.907 min of essentially 100 area % purity as shown in FIG. 5B. ¹H NMR (300 MHz, MeOH-d₄): δ 4.35 (dt, J=9.9 and 7.4 Hz, 8H), 4.10 (d, J=6.2 Hz, 1H), 3.91 (m, 1H), 3.82 (s, 3H), 3.21 (d, J=6.9 Hz), 2.84 (s, 3H), 2.27-2.47 (m, 2H), 2.03-2.22 (m, 4H), as shown in FIG. 6.

### Example 4. Preparation of Cocaine Base from EME HCl

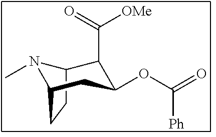

A glass reactor was charged with chloroform (amylene stabilized, 14.2 L), EME*HCl (1.53 kg, 6.51 mol), triethylamine (2.32 kg, 23.0 mol) and calcium oxide (552 g). The mixture was stirred for 30 min before benzoyl chloride (2.30 kg. 16.4 mol) was added. The resulting reaction mixture was stirred at 25° C. for 3.5 h. More triethylamine (0.459 kg, 4.54 mol) and benzoyl chloride (0.460 kg. 3.27 mol) were added and the reaction mixture stirred for another 12 h. At this point, GC analysis revealed a conversion of 95.4% of EME. Full conversion (99.8%, GC) of EME was reached after more triethylamine (0.506 kg, 5.00 mol) and benzoyl chloride (0.500 kg, 3.56 mol) were added and the reaction mixture stirred for 8 h.

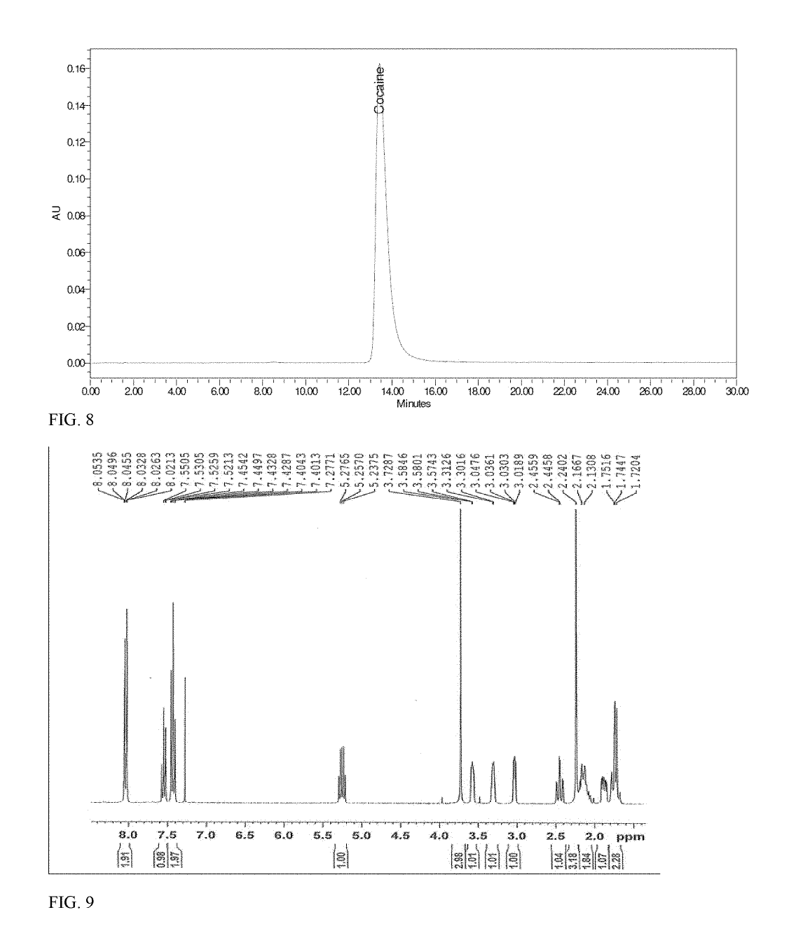

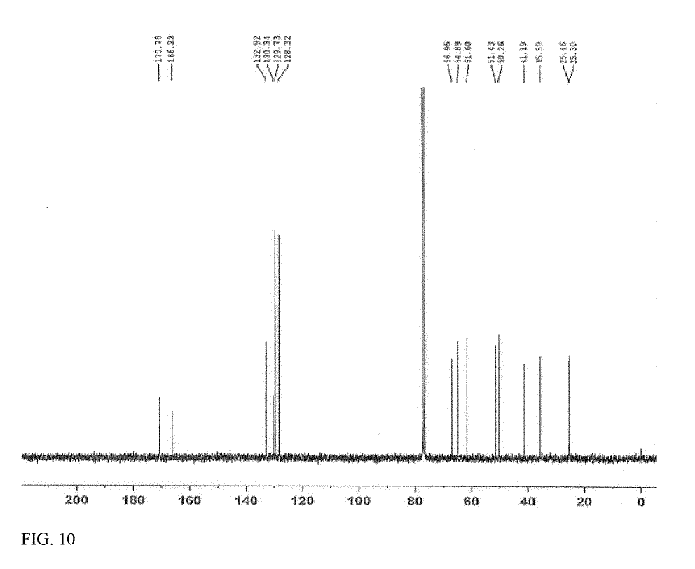

The reaction mixture was cooled to 11° C. and quenched slowly with a solution of conc HCl (10.2 mol/kg, 3.87 kg, 39.5 mol) in water (34 L) while the temperature was maintained below 35° C. The biphasic mixture was stirred for 12 min and allowed to settle for 20 min. The bottom layer was separated and extracted with water (9 L×2). The top layer (pH 1.0) was combined with the aqueous extracts and washed with chloroform (7 L×2). MTBE (23 L) was added to the aqueous layer. The resulting mixture was treated with ammonium hydroxide (27-30%, 12 L) and stirred vigorously for 5 min. Aqueous NaCl (30%, 9 L) was then added and the biphasic mixture stirred vigorously for 2 min. The bottom aqueous layer was separated and the top organic layer washed with aqueous NaCl (30%, 9 L). The combined aqueous layers were extracted with MTBE (10 L×2). The combined organic layers were washed with aqueous NaCl (30%, 9 L), cooled to 15° C. and treated with a solution of glacial acetic acid (5 L) in water (17 L). After stirring for 2 min, the bottom aqueous layer was separated and the top organic layer extracted with water (9 L×2). The combined aqueous layers were cooled to 18° C., diluted with isopropanol (4 L) and treated under stirring with ammonium hydroxide (27-30%, 12 L). The resulting slurry was stirred at rt for 30 min, transferred to a Buchner funnel and filtered. The crude cocaine thus obtained was treated with a solution of glacial acetic acid (1.5 L) in water (25 L) and stirred for 5 min until all solids were dissolved. The crude cocaine solution was treated with activated carbon by circulating and then filtering through a carbon capsule filter. The filtrate was cooled to 18° C., diluted with isopropanol (4 L) and treated slowly with ammonium hydroxide (27-30%, 7 L) under stirring. The resulting slurry was stirred at rt for 35 min. The solids were filtered and washed with water (7.5 L×3) to give 1.51 kg (yield: 76.6%) pure cocaine base as a white powder after drying under vacuum. HPLC revealed a purity of >99.5%. ¹H-NMR (300 MHz, CDCl₃): δ 8.02-8.05 (m, 2H), 7.55 (tt, J=1.4, 7.4 Hz, 1H), 7.43 (tm, J=7.3 Hz, 2H), 5.26 (td, J=5.9, 11.8 Hz, 1H), 3.73 (s, 3H), 3.56-3.59 (m, 1H), 3.30-3.32 (m, 1H), 3.03 (dd, J=3.4 and 5.2 Hz, 1H), 2.45 (dt, J=3.4, 11.8 Hz, 1H), 2.24 (s, 3H), 2.06-2.21 (m, 2H), 1.85-1.92 (m, 1H), 1.67-1.78 (m, 2H). ¹³C-NMR (75 MHz, CDCl₃): δ 25.3, 25.5, 35.6, 41.2, 50.3, 51.4, 61.6, 64.9, 67.0, 128.3, 129.7, 130.3, 132.9, 166.2, 170.8. HPLC chromatogram of cocaine base is shown in FIG. 8. Proton NMR spectrum of cocaine base in CDCl₃ is shown in FIG. 9. ¹³C-NMR spectrum of cocaine base in CDCl₃ is shown in FIG. 10.

### Example 5. Preparation of Cocaine Hydrochloride from Cocaine Base

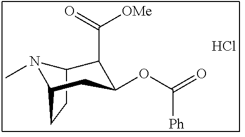

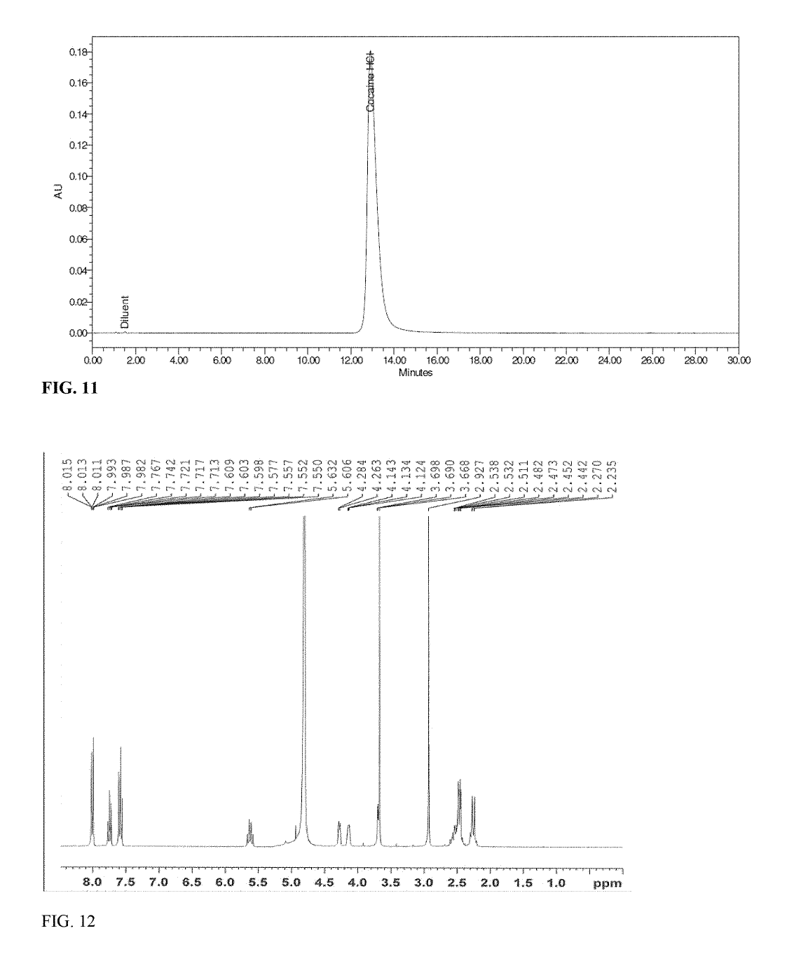

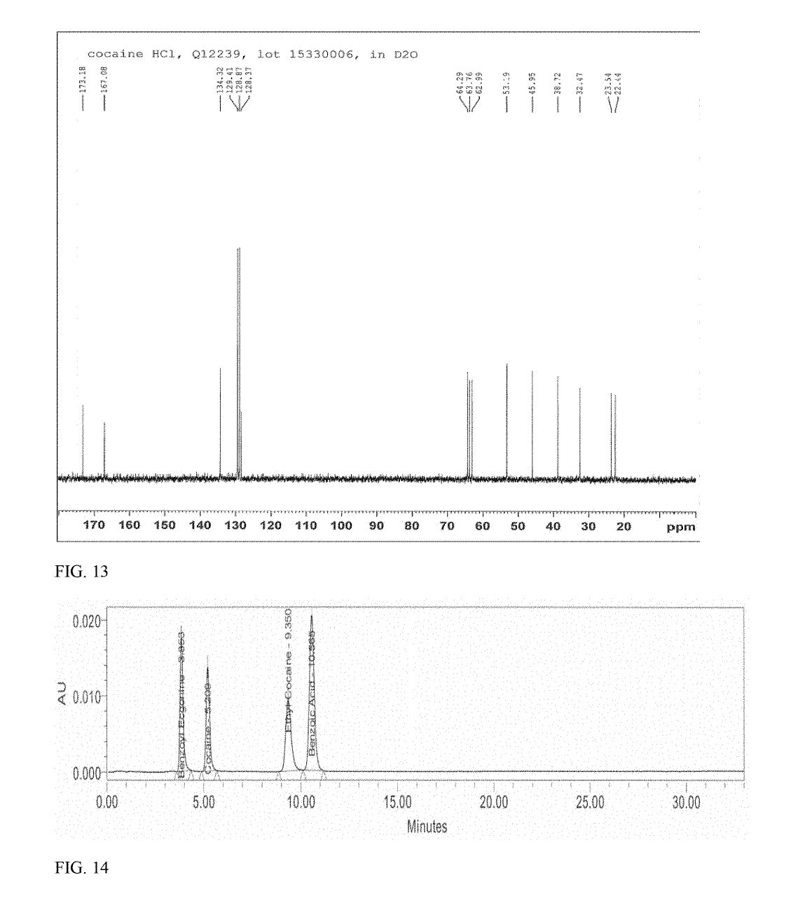

A glass reactor was charged with acetone (6.0 L) and cocaine base (1.01 kg, 3.33 mol). The solution was stirred at 20° C. while a solution of conc hydrochloric acid (0.333 kg, 10.2 mol/kg, 3.40 mol) in acetone (3.3 L) was added slowly over a period of 3 h. The resulting slurry was stirred at rt for 33 min. The solids were filtered and washed with acetone (1.6 L×3) to give 1.07 kg (yield: 94.5%) cocaine hydrochloride as a white powder after drying under vacuum. HPLC revealed a purity of >99.5%. ¹H-NMR (300 MHz, D₂O). δ 7.98-8.02 (m, 2H), 7.41 (tt, J=1.5, 7.5 Hz, 1H), 7.55-7.61 (m, 2H), 5.62 (q, J=8.5 Hz, 1H), 4.27 (bd, J=6.3 Hz, 1H), 4.12-4.16 (m, 1H), 3.68 (m, 1H), 3.67 (s, 3H), 2.93 (s, 3H), 2.40-2.60 (m, 4H), 2.20-2.30 (m, 2H). ¹³C-NMR (75 MHz, D₂O). δ 22.4, 23.5, 32.5, 38.7, 46.0, 53.2, 63.0, 63.8, 64.3, 128.4, 128.9, 129.1, 134.3, 167.1, 173.2. Specific rotation: [α]²⁵D-71.7° (c=2.0, H₂O). HPLC chromatogram of ethyl cocaine-free cocaine hydrochloride is shown in FIG. 11. Proton NMR spectrum of ethyl cocaine-free cocaine hydrochloride in D₂O is shown in FIG. 12. 13C-NMR spectrum of ethyl cocaine-free cocaine hydrochloride in D₂O is shown in FIG. 13.

### Example 6. HPLC Method A

HPLC Method A was employed to evaluate EME HCl. HPLC Method A employs stationary phase column Partisil™ SCX (Hichrom Limited), a strong cation-exchange stationary phase based on benzenesulphonic acid groups 10 μm, 4.6×250 mm. The Mobile Phase for HPLC Method A was *Buffer Solution: ACN (70:30), using isocratic elution, with a Column Temperature: 30° C., and a Sample Temperature: 5° C., injection volume 5 μL, Flow Rate: 1.0 mL/min, and with eluate monitored at Wavelength: 210 nm.

The *Buffer Solution Preparation was performed as follows. Accurately weigh about 6.8 g potassium phosphate monobasic into 1 L of water. Mix well to dissolve. Add 1.0 mL of triethylamine and mix well. Adjust pH to 4.0±0.05 using phosphoric acid. Potassium phosphate monobasic concentration is approximately 0.05M.

### Example 6B. HPLC Method B

Cocaine hydrochloride and related substances may be examined by liquid chromatography per European Pharmacopoeia 7.0-2, 2009, Monograph for cocaine hydrochloride (2.2.29).

Related Substances.

Examine by liquid chromatography (2.2.29).

Test Solution.

Dissolve 25.0 mg of the substance to be examined in the mobile phase and dilute to 50.0 mL with the mobile phase.

Reference Solution (a).

Dilute 1.0 mL of the test solution to 50.0 mL with the mobile phase. Dilute 5.0 mL of this solution to 100.0 mL with the mobile phase.

Reference Solution (b).

Dissolve 25 mg of the substance to be examined in 0.01 M sodium hydroxide and dilute to 10.0 mL with the same solvent. Dilute 1.0 mL of the solution to 10.0 mL with 0.01 M sodium hydroxide. Allow the solution to stand for 15 min.

Column:—size: 1=0.15 m, 0=4.6 mm, —stationary phase: end-capped octadecylsilyl silica gel for chromatography R (5 μm) with a specific surface area of 335 m²/g, a pore size of 10 nm and a carbon loading of 19.1 percent, —temperature: 35° C.

Mobile phase: triethylamine R, tetrahydrofuran R, acetonitrile R, water R (0.5:100:430:479.5 V/V/V/V).

Flow Rate:

1 mL/min.

Detection:

spectrophotometer at 216 nm.

Injection:

20 μL.

Relative retention with reference to cocaine (retention time=about 7.4 min): degradation product=about 0.7.

System Suitability:

reference solution (b):—resolution: minimum of 5 between the peaks due to cocaine and to the degradation product.

Limits:
any impurity eluting after the principal peak: not more than the area of the principal peak in the chromatogram obtained with reference solution (a) (0.1 percent),
total: not more than 5 times the area of the principal peak in the chromatogram obtained with reference solution (a) (0.5 percent),
disregard limit: 0.5 times the area of the principal peak in the chromatogram obtained with reference solution (a) (0.05 percent).

### Example 6C. HPLC Method for Related Substances in Naturally-Derived Cocaine Hydrochloride

Related substances in naturally-derived cocaine hydrochloride commercial samples were analyzed by the following HPLC method. Related substances include 2-furoyl ecgonine methyl ester, benzoyl ecgonine, ethyl cocaine, and benzoic acid, as shown in Table 6. A Phenomenex Synergi Hydro-RP, 4 m, 4.6×150 mm C18 polar endcapped reverse phase column was employed.

Buffer was prepared as follows. Dissolve 9.2 g of sodium phosphate monobasic monohydrate in 1000 mL of water. Sodium phosphate monobasic monohydrate concentration is approximately 0.067 M. Mobile phase was prepared as follows. For every 1 liter of mobile phase, thoroughly mix 650 mL of buffer with 350 mL of methanol. Add 1 mL of triethylamine. Allow the solution to reach room temperature before adjusting the pH. Adjust the pH to 3.00±0.05 with phosphoric acid. Filter using 0.45 m nylon filter under vacuum.

Stock solutions for sodium benzoate, ethylcocaine, and benzoyl ecgonine are prepared for the Resolution Solution. Resolution Solution for the Cocaine HCl is prepared.

Mobile phase of buffer:methanol:TEA (65:35:0.1) is employed as provided above with a column temperature of 30° C. and a sample temperature of 5° C. Flow rate was 1.5 mL/min and elution was monitored by UV at 230 nm. A 10 μL injection volume was employed.

Elution information is shown in Table 6.

TABLE 6
Related Substances in naturally-derived Cocaine HCl

| Component | ~RRT | RRF | (1/RRF)* |
|---|---|---|---|
| Cocaine HCl | 1.0 | 1.0 | 1.0 |
| 2-Furoyl Ecgonine Methyl | 0.6 | 1.0 | 1.0 |
| Ester | | | |
| Benzoyl Ecgonine | 0.75 | 1.1636 | 0.85940 |
| Ethyl Cocaine | 1.77 | 1.0749 | 0.93032 |
| Benzoic Acid | 2.13 | 2.4830 | 0.40274 |

RRT is relative retention time compared to cocaine hydrochloride.
RRF = relative response factor.
*(1/RRF) value is for entering into Empower Processing Method for proper calculation.

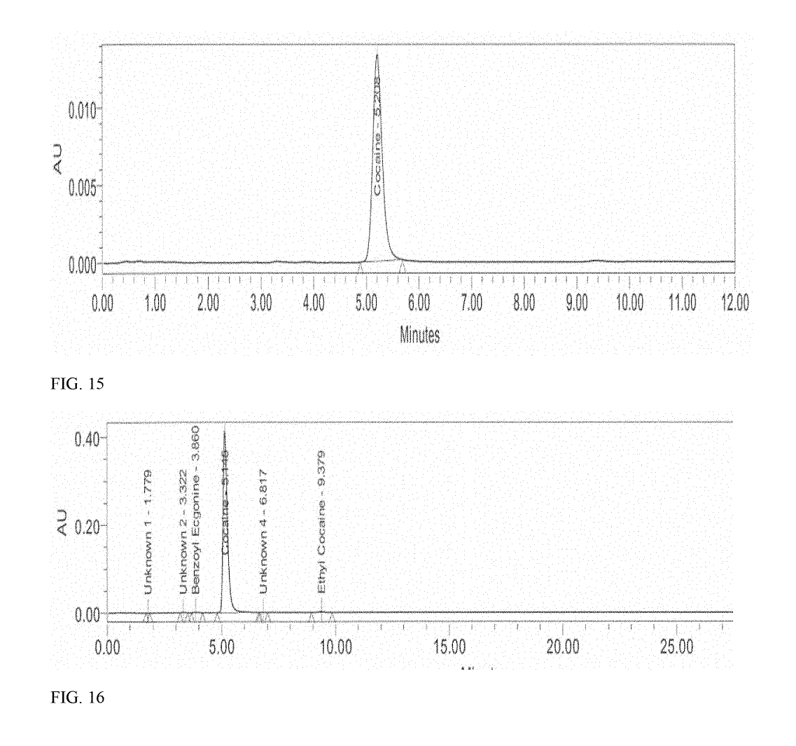

The limit of detection for ethyl cocaine in this method is 100 ppm (0.01%). Representative chromatograms for resolution standard solution, example standard cocaine and example naturally-derived sample are shown in FIGS. 14, 15 and 16, respectively. FIG. 16 shows chromatogram at 230 nm for representative sample of naturally-derived cocaine hydrochloride using HPLC method of Example 6C showing visible ethyl cocaine impurity at about 9.379 min retention time.

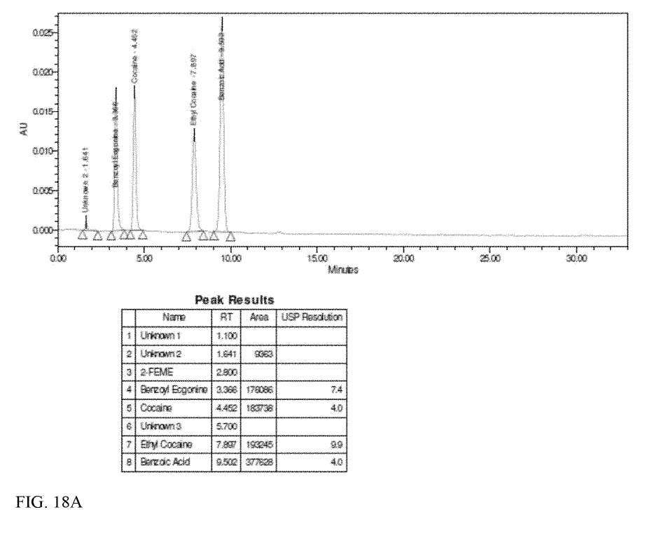

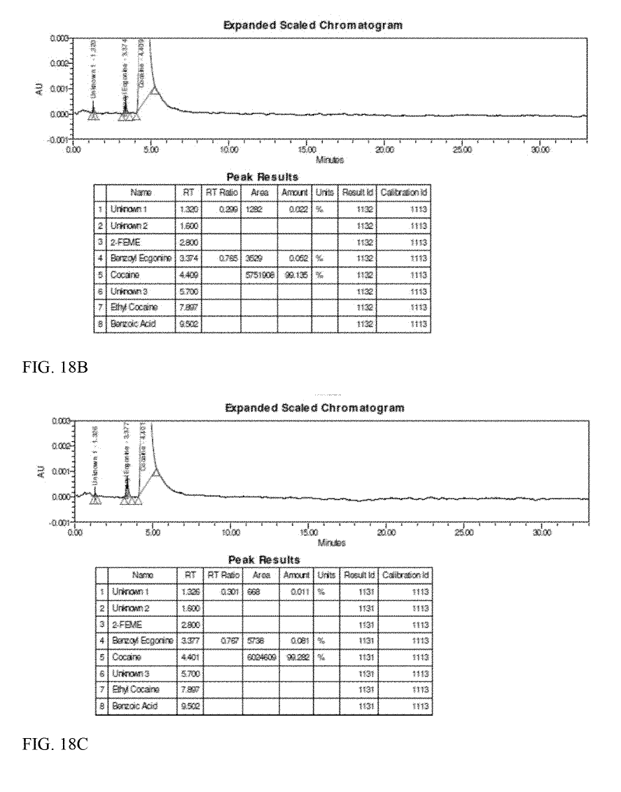

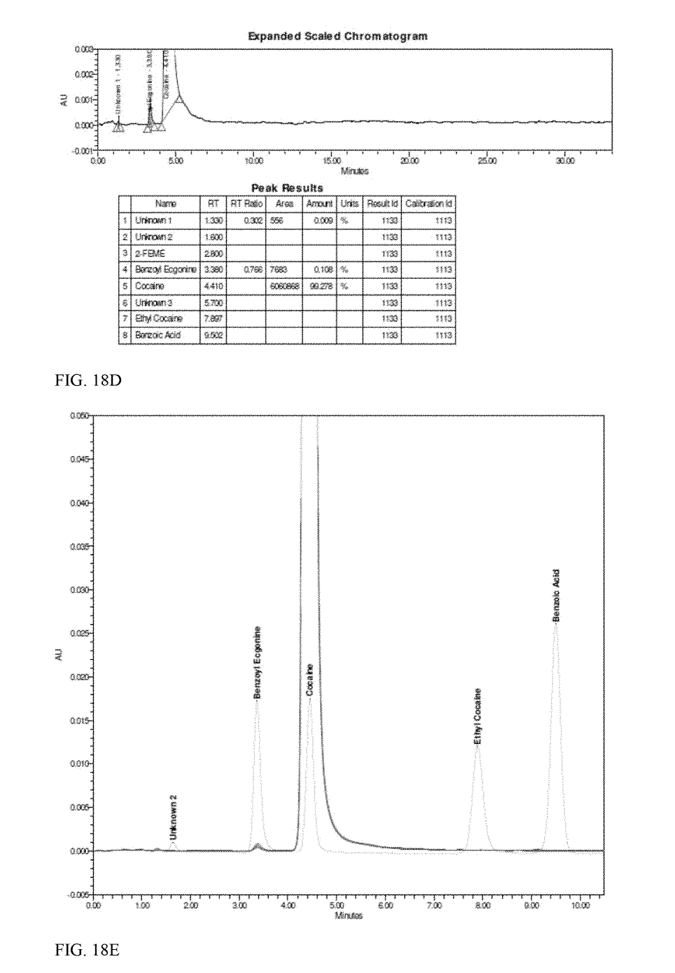

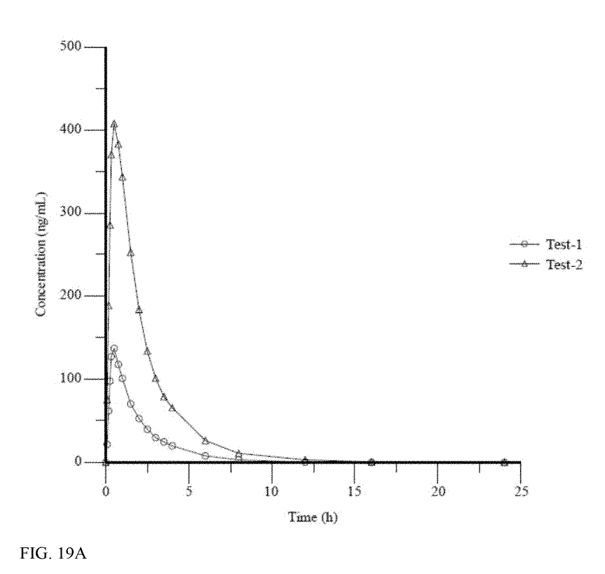

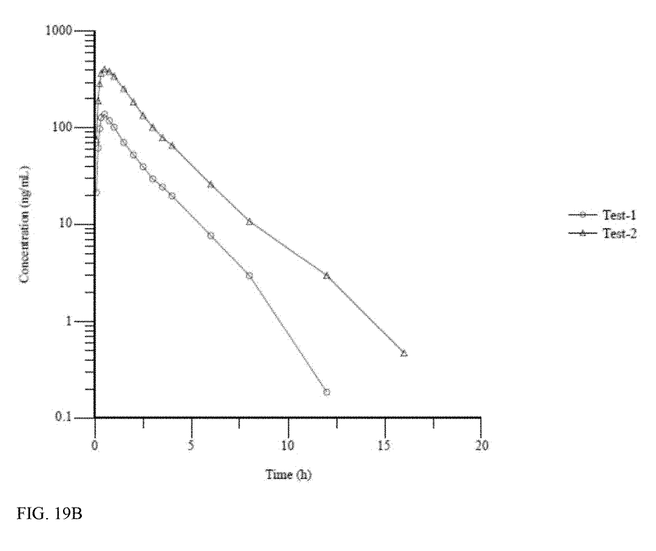

FIG. 18A shows a resolution chromatogram at 230 nm for representative resolution standard solution for related substances in cocaine hydrochloride HPLC method of Example 6C. FIGS. 18B, C and D show expanded scaled chromatograms at 230 nm of representative synthetic cocaine hydrochloride lots −859, −860, and −211 prepared according to the sodium amalgam method of the disclosure, by HPLC method of Example 6C, showing absence of detectable ethyl cocaine. FIG. 18E shows overlay chromatogram at 230 nm of resolution standard solution, and three representative lots of synthetic cocaine hydrochloride-859, −860 and −211, by HPLC method of Example 6C, showing absence of detectable ethyl cocaine. The three lots of isolated cocaine hydrochloride were shown to be ethyl cocaine-free.

### Example 6D. HPLC Method for Related Substances in Synthetically-Derived Cocaine Hydrochloride

Related substances in synthetically-derived cocaine hydrochloride samples prepared according to the disclosure were analyzed by the following HPLC method. Related substances in this method include benzoyl ecgonine, racemic benzoyltropine, dehydrobenzoyltropine, pseudococaine HCl, benzoic acid, and dehydrococaine, as shown in Table 7.

A Phenomenex Synergi Hydro-RP, 4 μm, 4.6×150 mm C18 polar endcapped reverse phase column was employed.

Buffer solution was prepared as follows. Weigh about 9.2 g of sodium phosphate monobasic monohydrate into 1000 mL of water. Dissolve and mix well. Add 1.0 mL of triethylamine and adjust the pH to 2.5±0.05 with phosphoric acid. Sodium phosphate monobasic monohydrate concentration is approximately 0.067 M.

Mobile phase was prepared by combining 760 mL of buffer solution with 240 mL of methanol and mixing well. Filter by vacuum using a 0.45 m nylon filter.

Analysis was run using 76:24 v/v buffer:methanol with a column temperature of 30° C. and a sample temperature of 5° C. Flow rate was 1.5 mL/min and elution was monitored by UV at 230 nm. A 10 μL injection volume was employed. Approximate elution time for cocaine hydrochloride was 12 minutes for cocaine. Additional analytes are shown in the Table 7 below.

TABLE 7
Related Substances in synthetically-derived Cocaine HCl

| Component | ~RRT | RRF | 1/RRF* |
|---|---|---|---|
| Benzoyl Ecgonine | 0.60 | 0.884096 | 1.131099 |
| Racemic Benzoyltropine | 0.91 | 1.376478 | 0.726492 |
| Cocaine HCl | N/A | N/A | N/A |
| Dehydrobenzoyltropine | 1.27 | 1.393714 | 0.717507 |
| Pseudococaine HCl | 1.49 | 0.951507 | 1.050964 |
| Benzoic Acid | 1.62 | 2.502025 | 0.399676 |
| Dehydrococaine | 1.77 | 1.539326 | 0.649635 |

RRT is relative retention time compared to cocaine hydrochloride.
RRF = relative response factor.
*(1/RRF) value is for entering into Empower Processing Method for proper calculation.

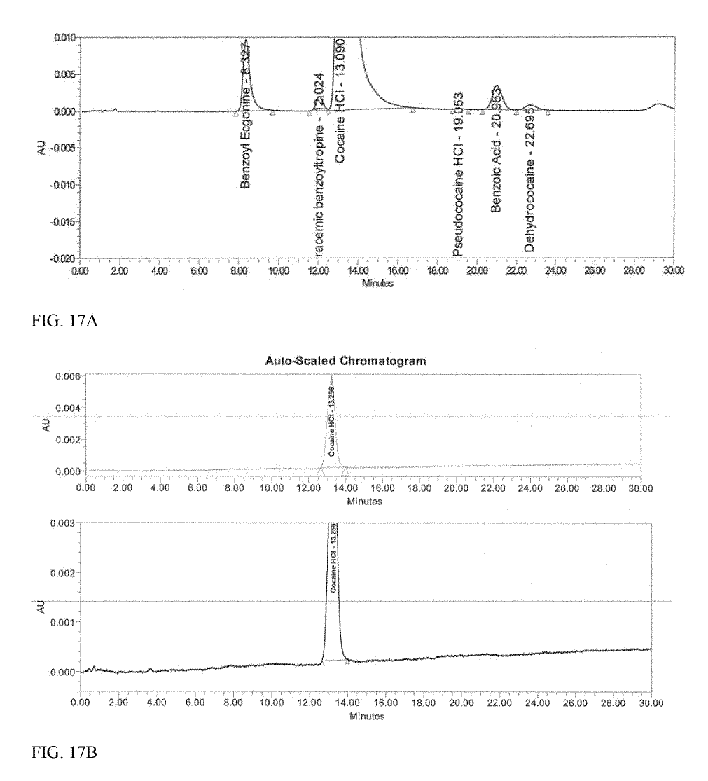

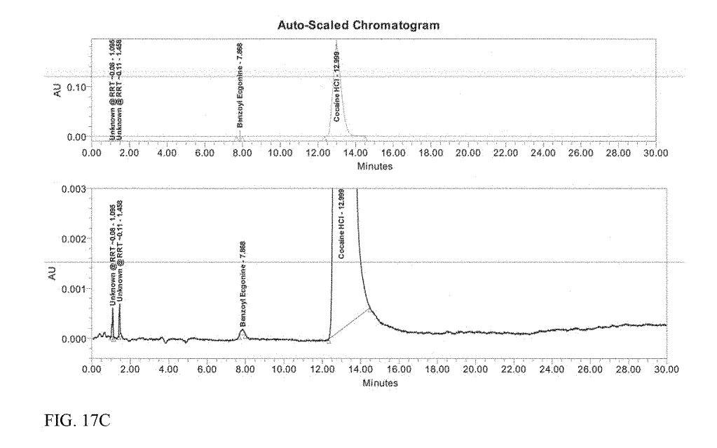

Representative chromatograms for resolution standard solution, example standard cocaine and example synthetically-derived sample are shown in FIGS. 17A, 17B and 17C, respectively.

### Example 7. Sample Preparation for GC Analysis

Analysis for certain intermediates or residual solvents was performed by Gas Chromatogaphy (GC) analysis. In particular, for residual solvents, a headspace gas chromatographic (GC) method using a flame ionization detector (FID) is employed using Restek Rtx-502.2, 60 m×0.53 mm×3.0 m, or equivalent. Dimethylsulfoxide was used as diluent, Helium was employed as carrier gas. Make-up gas and flow was helium or nitrogen, ˜30 mL/min. Oxidizer gas and flow was air, ˜400 mL/min; carrier flow of ˜3.0 mL/min using a split ratio of 5:1, and a split inlet liner with 1 mm ID. Injection volume was 1.0 mL, inlet temperature 190° C.; Detector temperature of 260° C. and run time of 32 minutes. Headspace sample parameters include oven temperature of 80° C., transfer line temperature 105° C., sample loop temperature 95° C.; vial equilibrium time of 10 min; GC cycle time of >42 min; vial pressurization of 1.0 min; loop fill time 0.30 min; loop equilibration 0.30 min; injection time 0.20 min; and vial pressure 18 psi. A gradient temperature program is shown in Table 8.

TABLE 8
GC Temperature Program

| | Ramp | Temperature | | Hold Time |
|---|---|---|---|---|
| Gradient | — | 35° | C. | 9.0 min |
| | 10° C./min | 45° | C. | 3.0 min |
| | 10° C./min | 50° | C. | 5.0 min |
| | 15° C./min | 125° | C. | 0.0 min |
| | 25° C./min | 200° | C. | 7.0 min |

Sample preparation for GC purity assay of intermediates and impurities including 2-CMT, EME, and PEM was performed as follows: EME HCl (10 mg) was suspended in CH₂Cl₂ (2 mL) and aq. 0.05 M Na₂CO₃ solution (0.8-1 mL) was added. The mixture was vigorously shaken for 20 sec. The organic layer was separated and the aqueous layer was back extracted with CH₂Cl₂ (2 mL). The combined organic layer was filtered through a pipette with a cotton plug and anhydrous K₂CO₃. A 1 μL aliquot (7-10 mg/l mL CH₂Cl₂) of the organic layer was injected to the gas chromatograph.

### Example 8. Comparison of Release Results for Naturally-Derived and Synthetically-Derived Cocaine Hydrochloride

A comparison of release results for Impurities for commercial naturally-derived Cocaine Hydrochloride, USP and synthetically-derived Cocaine Hydrochloride, USP prepared according to the present application by HPLC analysis of Examples 6C and 6D, respectively, is provided in this example. The comparative naturally-derived Cocaine Hydrochloride, USP was obtained from a commercial source and used in the comparative example below. Results are shown in Tables 9-12.

TABLE 9
Shared Impurities

| | Comparative | Inventive |
|---|---|---|
| | Natural | Synthetic |
| Impurity | Mean (%) +/− 3SD (%) | |
| Benzoic Acid (NMT 0.5%, | 0.00 +/− 0.01 | 0.00 +/− 0.00 |
| NMT 0.15% Synthetic) | | |
| Benzoyl Ecgonine (NMT | 0.05 +/− 0.10 | 0.05 +/− 0.14 |
| 0.5%) | | |
| Total Impurities (NMT 2.5% | 0.6 +/− 0.4 | 0.1 +/− 0.2 |
| Natural; NMT 2.0% | | |
| Synthetic) | | |
| Limit of Cinnamyl-Cocaine | Conforms | Conforms |
| and Other Reducing Substances¹ | | |
| Limit of Isoatropyl-Cocaine¹ | Conforms | Conforms |

¹This is a qualitative, color-change test that does not generate numerical results.

The synthetic cocaine hydrochloride prepared according to the present disclosure exhibited not more than 0.15%, not more than 0.1%, or not more than 0.05% benzoic acid by HPLC. The synthetic cocaine hydrochloride prepared according to the present disclosure exhibited not more than 0.5%, not more than 0.1%, or not more than 0.07% benzoyl ecgonine by HPLC. The synthetic cocaine hydrochloride prepared according to the present disclosure exhibited not more than 2.0%, not more than 1.0%, not more than 0.5%, not more than 0.3%, or not more than 0.2% total impurities by HPLC. Specifically, the synthetic cocaine hydrochloride prepared according to the present disclosure exhibited not more than 0.005% benzoic acid, not more than 0.1% benzoyl ecgonine, and not more than 0.2% total impurities when tested according to HPLC protocol of Example 6D for cocaine hydrochloride, as shown in Table 9.

TABLE 10
Unshared Impurities

| | Comparative | Inventive |
|---|---|---|
| | Natural | Synthetic |
| Impurity | Mean (%) +/− 3SD (%) | |
| 2-Furoyl Ecgonine Methyl | 0.00 +/− 0.00 | N/A |
| Ester (2-FEME) (NMT 0.5%) | | |
| Ethyl Cocaine (NMT 2.0%) | 0.49 +/− 0.52 | N/A |
| Ecgonine (NMT 0.15%) | N/A | 0.00 +/− 0.00 |
| EME (NMT 0.5%) | N/A | 0.00 +/− 0.00 |
| Pseudococaine (NMT 0.15%) | N/A | 0.00 +/− 0.00 |
| Dehydrococaine (NMT 0.15%) | N/A | 0.00 +/− 0.00 |
| Benzoylpseudotropine | N/A | 0.00 +/− 0.00 |
| (NMT 0.15%) | | |
| Dehydrobenzoyltropine | N/A | 0.00 +/− 0.02 |
| (NMT 0.15%) | | |

N/A refers to not applicable per route of synthesis, thus not tested.

The synthetic cocaine hydrochloride prepared according to the present disclosure exhibited not more than 0.01% ethyl cocaine. The synthetic cocaine hydrochloride prepared according to the present disclosure exhibited not more than 0.15%, not more than 0.1%, not more than 0.05%, or not more than 0.01% ecgonine. The synthetic cocaine hydrochloride prepared according to the present disclosure exhibited not more than 0.5%, not more than 0.1%, not more than 0.05%, or not more than 0.01% EME. The synthetic cocaine hydrochloride prepared according to the present disclosure exhibited not more than 0.15%, not more than 0.1%, not more than 0.05%, or not more than 0.01% pseudococaine. The synthetic cocaine hydrochloride prepared according to the present disclosure exhibited not more than 0.15%, not more than 0.1%, not more than 0.05%, or not more than 0.01% dehydrococaine. The synthetic cocaine hydrochloride prepared according to the present disclosure exhibited not more than 0.15%, not more than 0.1%, not more than 0.05%, or not more than 0.01% benzoylpseudotropine. The synthetic cocaine hydrochloride prepared according to the present disclosure exhibited not more than 0.15%, not more than 0.1%, not more than 0.05%, or not more than 0.01% 2-CMT. The synthetic cocaine hydrochloride prepared according to the present disclosure exhibited not more than 0.15%, not more than 0.1%, not more than 0.05%, or not more than 0.01% PEM. The synthetic cocaine hydrochloride prepared according to the present disclosure exhibited not more than 0.15%, not more than 0.1%, not more than 0.05%, or not more than 0.01% dehydrobenzoyltropine, when tested according to HPLC method of Example 6D.

Specifically, the synthetic (−)-cocaine hydrochloride prepared according to the present disclosure exhibited not more than 0.15% (+)-cocaine HCl, not more than 0.15% pseudococoaine, not more than 0.15% dehydrococaine, not more than 0.15% benzoic acid, not more than 0.5% benzoyl ecgonine, not more than 0.15% racemic benzoyltropine, not more than 0.15% dehydrobenzoyltropine, not more than 0.10% each unknown related substance, not more than 0.15% ecgonine, not more than 0.5% methyl ecgonine, not more than 0.15% 2-CMT, not more than 0.15% PEM, and not more than 1.0% total impurities, when tested according to USP protocols for cocaine hydrochloride as shown in Table 10. In contrast, the naturally-derived cocaine hydrochloride exhibited 0.49+/−0.52% ethyl cocaine.

### Example 9. Cocaine Hydrochloride Pharmaceutical Compositions-Solutions

Cocaine Hydrochloride solutions were prepared for topical application using the (−)-cocaine hydrochloride of Example 5. Formulations are shown in Tables 13 and 14 below. The topical solution is in a range from pH 3.0 to 4.2.

TABLE 11
Cocaine HCl Topical Solution, 4%

| | Concentration | g per batch | Concentration |
|---|---|---|---|
| Ingredient | (mg/mL) | (30 L) | (wt/v) |
| Cocaine HCl, ethyl | 40 | 1,200.0 | 4.00% |
| cocaine-free | | | |
| Sodium Benzoate, NF | 1.0 | 30.0 | 0.10% |
| D & C Yellow #10 | 0.0044 | 0.132 | |
| FD & C Green #3 | 0.0043 | 0.129 | |
| Citric Acid Anhydrous, | 1.33 | 40.0 | 0.133% |
| USP | | | |
| Purified Water, USP | Q.S. | Q.S. | |

TABLE 12
Cocaine HCl Topical Solution, 10%

| | Concentration | g per batch | Concentration |
|---|---|---|---|
| Ingredient | (mg/mL) | (10 L) | (wt/v) |
| Cocaine HCl, ethyl | 100 | 1,000.0 | 10.0% |
| cocaine-free | | | |
| Sodium Benzoate, NF | 1.0 | 10.0 | 0.10% |
| D & C Yellow #10 | 0.0044 | 0.044 | |
| FD & C Green #3 | 0.0043 | 0.043 | |
| Citric Acid Anhydrous, | 1.33 | 13.3 | 0.133% |
| USP | | | |
| Purified Water, USP | Q.S. | Q.S. | |

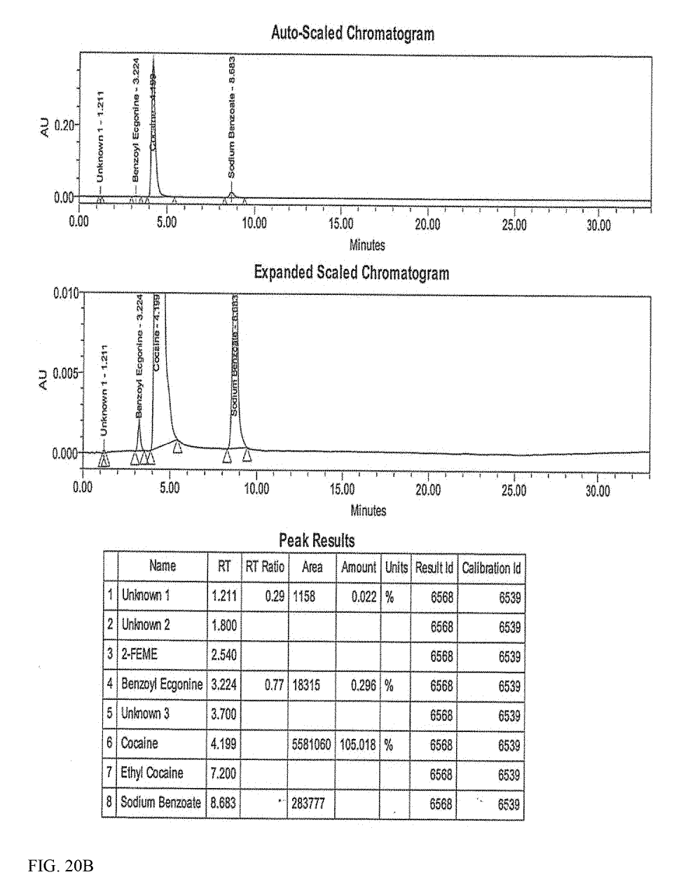

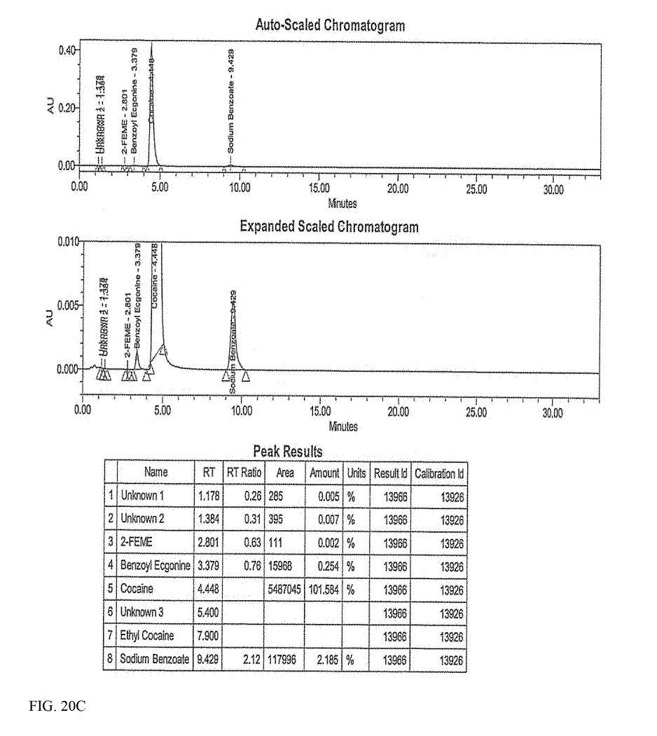

The compositions of Tables 11 and 12 included ethyl cocaine-free cocaine hydrochloride having no more than 0.01% ethyl cocaine. FIG. 20A shows an HPLC chromatogram of a resolution solution including benzoyl ecgonine, cocaine, ethyl cocaine, and sodium benzoate monitored at 230 nm. The HPLC method was validated to a LOD of 0.01% and a LOQ of 0.05%. FIG. 20B shows HPLC analysis of a representative Cocaine HCl Topical Solution, 4% w/v, according to Table 11. FIG. 20C shows HPLC analysis of a representative Cocaine HCl Topical Solution, 10% w/v, according to Table 12. FIGS. 20A and 20B HPLC chromatograms provide evidence of absence of detectable ethyl cocaine in representative drug product.

### Example 10. Clinical Trials

Cocaine HCl is a local anesthetic, which binds to and blocks the voltage-gated sodium channels in the neuronal cell membrane. Cocaine produces potent sympathomimetic effects by increasing norepinephrine concentrations in postsynaptic receptors by inhibiting presynaptic reuptake. Cocaine HCl blocks the initiation or conduction of nerve impulses following local application. When applied topically to mucous membranes, the drug produces a reversible loss of sensation and vasoconstriction.

A total of 670 subjects in 3 clinical studies (two Phase 3 randomized placebo-controlled Clinical Trials and 1 Pharmacokinetic study) were treated with Cocaine Hydrochloride Topical Solution; including 352 subjects treated with the 4% solution (single 160 mg dose), and 354 subjects treated with the 10% solution (single 400 mg dose). In the two Phase 3 trials a single topical dose of Cocaine Hydrochloride Topical Solution, 4% or 10%, was administered according to Tables 11 and 12.

Study 1 was a Phase 3, multicenter, randomized, double-blind, placebo controlled, parallel-groups study designed to compare the efficacy and safety of intranasally administered Cocaine HCl Topical Solution, 4% and 10%, to placebo for providing adequate anesthesia to complete a nasal procedure or surgery.

A total of 120 patients were enrolled in ten clinical centers and randomized to one dose of cocaine HCl topical solution, 4% (n=39), cocaine HCl topical solution, 10% (n=41), or placebo (n=40) applied to the nasal mucosa for 20 minutes. All randomized patients completed the study nasal procedure or surgery.

The immediate and sustained analgesia success was significantly greater for the cocaine HCl 10% treatment group (253 mg mean dose) than for the placebo group, 75.6% versus 37.5%, respectively with a treatment difference of 38.1%, which was statistically (p=0.0005) and clinically significant.

The proportion of subjects with immediate and sustained analgesia success was not statistically significant between the cocaine HCl 4% treatment group (108 mg mean dose) and placebo group, 53.9% versus 37.5% (p=0.1088). Lack of a statistically significant difference was due in part to the unexpectedly high placebo response and use of a suboptimal nasal pressure-generating device (von Frey monofilament).

All patients in both active treatment groups had adequate hemostasis as assessed by the investigator.

Study 2 was a Phase 3, multicenter, randomized, double-blind, placebo controlled, parallel-groups study designed to compare the efficacy and safety of intranasally administered cocaine HCl topical solution, 4% and 10%, to placebo for providing adequate anesthesia to complete a nasal procedure or surgery.

A total of 646 patients were enrolled in twenty clinical centers and randomized to one dose of cocaine HCl topical solution, 4% (n=259), cocaine HCl topical solution, 10% (n=259), or placebo (n=128) applied to the nasal mucosa for 20 minutes. Two subjects in the cocaine HCl topical solution, 4% treatment group discontinued the study due to adverse event-related drug reasons and required early removal of the pledgets from their nasal cavities. Three subjects in the cocaine HCl topical solution, 10% treatment group required early removal of the pledgets from their nasal cavities but completed the study procedure or surgery.

Sixty-one percent (6.8%) of randomized patients were female and 8.8% were white, with a mean age was 37.6 years (range 18 to 76 years).

The immediate and sustained anesthesia success was significantly greater for the cocaine HCl topical solution, 4% treatment group (126 mg mean dose) than for the placebo group, 70.9% versus 19.7%, respectively, with a treatment difference of 51.2%, which was statistically significant (p<0.0001) and clinically significant.

A statistically and clinically significant difference was observed between the cocaine HCl topical solution, 10% treatment group (319 mg mean dose) and placebo group, with the proportion of patients demonstrating immediate and sustained anesthesia of 82.7% versus 19.7% (p<0.0001), respectively, with a treatment difference of 63.0%. An exploratory analysis demonstrated that a difference exists between the cocaine HCl 10% and cocaine HCl 4% treatments (p=0.0011).

Patients in both active treatment groups had adequate hemostasis, produced by cocaine's local nasal vasoconstriction, as assessed by the investigator.

When applied to mucous membranes by pledget administration, topical anesthesia develops rapidly and persists for 30 minutes or longer depending on the concentration of cocaine HCl solution used, the dose, and on the vascularity of the tissue.

### Example 11. Pharmacokinetic Studies

A single dose study was designed with the intent to characterize the pharmacokinetic behavior of cocaine and its metabolites (benzoylecgonine, ecgonine methyl ester. ecgonine, and norcocaine) in both plasma and urine, following administration of the study treatments in healthy subjects. Pharmacokinetic studies were performed using the formulations shown in Tables 11 and 12.

The study treatments (placebo, Test-1 and Test-2) were administered topically, in the nasal cavity as follows: For each administration, four pledgets were treated with 4 mL of the assigned solution (Test-1, Test-2 or placebo). The 4 mL treatment of the Test-1 (4% cocaine HCl solution) corresponded to a 160 mg dose of cocaine. The 4 mL treatment of the Test-2 (10% cocaine HCl solution) corresponded to a 400 mg dose of cocaine. Two pledgets were placed into each nostril (one pledget on the inner left side and one pledget on the inner right side of each nostril). The pledgets were retained in the nasal cavity for 20 minutes prior to being removed. Subjects remained seated for at least 1 hour following placement of the pledgets into the nasal cavity. The rayon pledgets (½″×3″ in size), were manufactured by DeRoyal No. 30-057.

The direct measurements of this study were the plasma and urine concentrations of cocaine and its metabolites (benzoylecgonine, ecgonine methyl ester, ecgonine, and norcocaine). These concentrations were obtained by analysis of the plasma derived from the blood samples drawn and from the urine collected during this study. For the plasma analysis, the experimental samples were assayed for cocaine and its metabolites (benzoylecgonine, ecgonine methyl ester, ecgonine, and norcocaine) using validated HPLC (High Performance Liquid Chromatography) methods with MS/MS (mass spectrometry/mass spectrometry) detection. The lower limit of quantitation and upper limit of quantitation for each analyte were as follows: Cocaine and benzoylecgonine assay range: 2.00 ng/mL to 650.00 ng/mL; Ecgonine Methyl Ester assay range: 1.00 ng/mL to 100.00 ng/mL; Ecgonine assay range: 0.500 ng/mL to 100.000 ng/mL; and Norcocaine assay range: 0.150 ng/mL to 100.000 ng/mL.

In a human adult, single-dose pharmacokinetic study, the application of Cocaine Hydrochloride Topical Solution, 4% (Test-1; n=33) and 10% (Test-2; n=30), for 20 minutes by pledgets produced nasal vasoconstriction significantly reducing capillary blood flow, assessed by laser Doppler perfusion. Statistical analysis showed that 160 mg (4 mL, 4%) and 400 mg (4 mL, 10%) cocaine HCl topical solution doses are significantly different from placebo (each comparison p<0.0001), suggesting reduced blood flow and increased vasoconstriction to the nasal mucous membranes.

Analysis of Efficacy

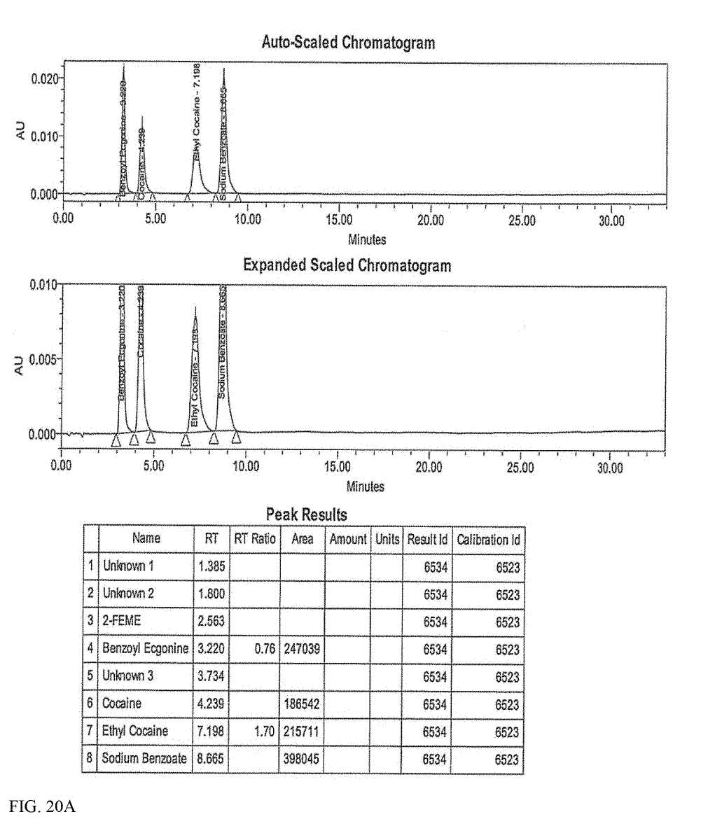

Mean plasma concentration-time profiles for cocaine are displayed by treatment in FIG. 19A (linear scale) and FIG. 19B (logarithmic scale). Plasma levels were below the lower limit of quantification (LOQ, 2.00 ng/mL) in all samples collected prior to dosing. The wash-out period between doses was considered appropriate.

Plasma pharmacokinetic parameter values by treatment are presented in Table 13.

TABLE 13
Summary of Plasma Cocaine Pharmacokinetic Parameters

| Parameter | Test-1 (n = 33) | | Test-2 (n = 30) | |
|---|---|---|---|---|
| (Units) | Mean | (C.V. %) | Mean | (C.V. %) |
| Cmax | 142.68 | (44.9) | 433.53 | (49.3) |
| (ng/mL) | | | | |
| ln (Cmax) | 4.8668 | (9.0) | 5.9804 | (7.0) |
| Tmax | 0.50 | (0.17-1.00) | 0.50 | (0.33-1.00) |
| (hours)a | | | | |
| AUC_{0-T} | 279.01 | (46.6) | 950.54 | (43.5) |
| (ngh/mL) | | | | |
| ln (AUC_{0-T}) | 5.5528 | (6.8) | 6.7761 | (5.9) |
| AUC_{0-∞} | 286.68 | (45.6) | 960.09 | (43.1) |
| (ng · h/mL) | | | | |
| ln (AUC_{0-∞}) | 5.5828 | (6.7) | 6.7874 | (5.9) |
| AUC 0-T/0-∞ (%) | 97.05 | (1.2) | 98.88 | (0.7) |
| λZ (hours-1) | 0.4576 | (13.7) | 0.3757 | (26.1) |
| Thalf (hours) | 1.54 | (13.5) | 2.01 | (36.8) |

A summary of the statistical analysis of Cmax and AUC for cocaine is given in Table 14.

TABLE 14
Summary of the Statistical Analysis of Cocaine

| | Intra- | Geometric LSmeans^{a} | | | 90% Confidence | |
|---|---|---|---|---|---|---|
| | Subject | Test-1 | Test-2 | Ratio | Limits (%) | |
| Parameter | C.V. (%) | (n = 33) | (n = 30) | (%) | Lower | Upper |
| Cmax | 28.4 | 129.48 | 389.99 | 33.20 | 29.41 | 37.49 |
| AUC_{0-T} | 26.6 | 257.35 | 869.29 | 29.61 | 26.42 | 33.17 |
| AUC_{0-∞} | 26.4 | 265.22 | 879.71 | 30.15 | 26.93 | 33.75 |

^{a}units are ng/mL for Cₘₐₓ and ng · h/mL for AUC_{0-T} and AUC_{0-∞}

The intra-subject variability reflects the residual variability observed in the pharmacokinetic parameters after accounting for possible differences between sequence, period, and formulation effects as well as accounting for between-subject variations. The intra-subject coefficients of variation were 28.4%, 26.6% and 26.4% for Cmax, AUC0-T, and AUC0-∞, respectively (Table 10). The intra-subject coefficients of variation were all below 30%, which indicates that the drug products are not highly variable.

The relationship between local anesthetic effectiveness and toxicity of cocaine is a function of the patient's state of health, medical condition, nasal mucosa integrity and extent of systemic absorption of cocaine (from the pledgets).

Absorption

Application of the topical cocaine hydrochloride solutions for 20 minutes by pledget administration to the nasal mucosa in healthy adults significantly minimizes the systemic absorption of the applied dose of cocaine HCl. The mean systemic absorption of cocaine from a single 160 mg dose (4 mL, 4%)(n=33) was 23.44% of the topically applied dose. The mean systemic absorption of cocaine from a single 400 mg dose (4 mL, 10%) (n=30) was 33.34% of the topically applied dose as shown in Table 15.

TABLE 15
Systemic Absorption in Healthy Adult Subjects Minimized
by Pledget Administration (single nasal dose of 160 mg and
400 mg Cocaine HCl Topical Solution over 20 minutes)

| Cocaine HCl | | | | | Median |
|---|---|---|---|---|---|
| Topical | | | | | Tₘₐₓ |
| Solution, | Age | Application | Estimated¹ | Mean | (min) |
| and Dose | Range | Time | Systemic | Cₘₐₓ | Cₘₐₓ |
| (4 mL) | (yr) | (min) | Absorption | (ng/mL) | (ng/mL) |
| 160 mg | 20-40 | 20 | 23.44% | 142.68 | 30 |
| (4%) | | | | n = 33 | 142.7 |
| 400 mg | 20-40 | 20 | 33.34% | 433.53 | 30 |
| (10%) | | | | n = 30 | 433.5 |

¹Estimated absorbed dose was calculated by subtracting the residual amount of drug in the pledgets from the administered dose;
Tₘₐₓ includes time 0 (the start of pledget insertion to pledget removal (20 minutes) to the time Cₘₐₓ was observed, i.e. 10 minutes after removal of the pledgets.

Distribution

Cocaine is extensively distributed to tissues and crosses the blood brain barrier. Its volume of distribution is approximately 2 L/kg. Cocaine crosses the placenta by simple diffusion, and accumulates in the fetus after repeated use.

Metabolism

Cocaine is metabolized by two major hydrolytic pathways. Cocaine (40-45%) is metabolized by hydrolysis to benzoylecgonine (major, but inactive metabolite) by hepatic carboxylesterase-1. Cocaine (40-45%) is also metabolized by hydrolysis to ecgonine methyl ester (major, but inactive metabolite) by plasma butyrylcholinesterase and hepatic carboxylesterase-2.

Cocaine is minimally metabolized by hydrolysis to ecgonine (minor, inactive metabolite) by carboxylesterase-2.

Cocaine (5-10%) is N-demethylated by the CYP3A4 enzyme system to produce the active metabolite, norcocaine. Total systemic exposure of norcocaine is less than one percent that observed with cocaine.

Excretion

Cocaine is excreted almost exclusively in the urine, as metabolites. Only a minor fraction of cocaine is eliminated unchanged in the urine (<5%).

The apparent elimination half-life (Thalf; mean+% CV) of cocaine following administration of Cocaine hydrochloride topical solutions (by pledgets) was 1.54 hours (+13.5) for the 4% concentration, and 2.10 hours (+36.8) for the 10% concentration. All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

We claim:

1. A method of preparing (−)-cocaine or a pharmaceutically acceptable salt thereof comprising:
exposing (+)-2-carbomethoxy-3-tropinone (2-CMT) or a salt thereof to sodium mercury amalgam (Na—Hg) and an inorganic acid in an aqueous solution whereby the 2-CMT or salt thereof is converted to a mixture of compounds comprising (−)-ecognine methyl ester ((−)-EME) and pseudoecgonine methyl ester (PEM); and
benzoylating the (−)-EME or a pharmaceutically acceptable salt thereof to form (−)-cocaine or a pharmaceutically acceptable salt thereof.

2. The method of claim 1, further comprising
separating the (−)-EME or pharmaceutically acceptable salt thereof from the PEM or a pharmaceutically acceptable salt thereof.

3. The method of claim 2, wherein the separating comprises dissolving the mixture of compounds comprising the (−)-EME and the PEM in isopropyl alcohol; adding methanolic HCl to form a solution mixture; and adding acetone to the solution mixture to form a heterogenous mixture, wherein (−)-EME HCl precipitates from the mixture.

4. The method of claim 2, wherein the separating comprises stirring the mixture of compounds comprising the (−)-EME and the PEM in cyclohexane, allowing the PEM to precipitate, and filtering off the precipitated PEM.

5. The method of claim 3, wherein the solution mixture is at least partially evaporated and fresh isopropyl alcohol is added prior to adding the acetone.

6. The method of claim 1, wherein at least 96% of the 2-CMT or salt thereof is converted to the mixture comprising (−)-EME and PEM as determined by GC area %.

7. The method of claim 1, wherein the 2-CMT or salt thereof is (+)-2-carbomethoxy-3-tropinone (2-CMT) bitartrate.

8. The method of claim 1, wherein the inorganic acid is sulfuric acid, wherein the sulfuric acid in the exposing step is employed to maintain the pH between 3.5 and 4.5.

9. The method of claim 8, wherein the temperature of the aqueous solution during the exposing step is maintained from 5-10° C.

10. The method of claim 8, wherein the (+)-2-carbomethoxy-3-tropinone bitartrate is exposed to the sodium mercury amalgam and the acid for a period of from 2 to 18 hours, to form the mixture of compounds comprising the (−)-EME and the PEM.

11. The method of claim 10, wherein the ratio of (−)-EME to PEM in the mixture is at least 1.3:1 or higher by GC area %.

12. The method of claim 1, wherein the exposing comprises continuously supplying sodium amalgam from an electrolyzing unit to the aqueous solution of (+)-2-carbomethoxytropinone or salt thereof and the acid; and continuously transferring spent amalgam from the reactor to the electrolyzing unit.

13. The method of claim 1, wherein the exposing step comprises allowing an insoluble sodium salt of the inorganic acid to form during the exposing step.

14. The method of claim 10, wherein the exposing step comprises adding a base to the mixture of compounds to increase the pH of the mixture to within a range from about pH 8.7 to pH 11.

15. The method of claim 1, wherein the pharmaceutically acceptable salt of (−)-cocaine is (−)-cocaine hydrochloride having not more than 0.15% ethyl cocaine, and not more than 1.0% total impurities by HPLC area %.

16. The method of claim 15, wherein the (−)-cocaine hydrochloride has not more than 0.01% ethyl cocaine, and one or more of the group consisting of not more than 0.15% (+)-cocaine hydrochloride, not more than 0.15% pseudococaine, not more than 0.15% dehydrococaine, not more than 0.15% benzoic acid, not more than 0.5% benzoyl ecgonine, not more than 0.15% benzoyltropine, not more than 0.15% dehydrobenzoyltropine, not more than 0.15% ecgonine, not more than 0.5% methylecgonine, not more than 0.15% 2-CMT, and not more than 0.15% PEM by HPLC area %.

17. The method of claim 1, wherein ethanol is not employed as a solvent.

18.-23. (canceled)

24. Isolated (−)-cocaine hydrochloride having not more than 0.15% ethyl cocaine.

25. The isolated (−)-cocaine hydrochloride of claim 24 having not more than 100 ppm ethyl cocaine.

26. A method for introduction of local anesthesia in a human subject in need thereof comprising administering a pharmaceutical composition comprising an effective amount of (−)-cocaine hydrochloride having not more than 0.15% ethyl cocaine, and a pharmaceutically acceptable carrier.

27. The method of claim 26, wherein the pharmaceutical composition comprises
2 to 20 wt % of the (−)-cocaine hydrochloride;
0.05-0.2 wt % sodium benzoate; and
0.05-0.2 wt % citric acid.

28. The method of claim 27, wherein the composition is administered prior to a surgery or a diagnostic procedure, wherein the administering comprises topically applying the composition to one or more mucous membranes in the subject, wherein the mucous membrane is selected from the group consisting of oral, laryngeal, and nasal mucous membranes.

29. The method of claim 28, wherein the (−)-cocaine hydrochloride having not more than 0.15% ethyl cocaine is prepared by a method according to claim 1.

30. The method of claim 28, wherein the mean systemic absorption is between 20% to 35% of the total administered dose of (−)-cocaine hydrochloride.
